(19) United States
(12) Patent Application Publication
(10) Pub. No.: US 2006/0210557 A1
(43) Pub. Date: Sep. 21, 2006

(54) Stabilized liquid polypeptide formulations

(76) Inventors: Donna Luisi, North Andover, MA (US); Nicholas W. Warne, Andover, MA (US); Angela Kantor, Pepperell, MA (US)

Correspondence Address: LAHIVE & COCKFIELD, 28 STATE STREET, BOSTON, MA, 02109, US

(21) Appl. No.: 11/342,252

(22) Filed: Jan. 27, 2006

Related U.S. Application Data
(60) Provisional application No. 60/648,639, filed on Jan. 28, 2005.

(51) Int. Cl. A61K 39/395; A61K 31/4172; A61K 31/198

(52) U.S. Cl. 424/133.1; 514/400; 514/562

(57) ABSTRACT

The present invention provides formulations for maintaining the stability of polypeptides, in particular, therapeutic antigen-binding polypeptides such as antibodies and the like, for example, anti-Aβ antibodies. The formulations generally include an antioxidant in a sufficient amount as to inhibit by-product formation, for example, the formation of high molecular weight polypeptide aggregates, low molecular weight polypeptide degradation fragments, and mixtures thereof. The formulations of the invention optionally comprise a tonicity agent, such as mannitol, and a buffering agent or amino acid such as histidine, and thus, the formulations are suitable for several different routes of administration.

### RELATED INFORMATION

This application claims the benefit of U.S. provisional patent application bearing Ser. No. 60/648,639 (filed Jan. 28, 2005), entitled “Stabilized Liquid Polypeptide Formulations.” The entire content of the above-referenced application is incorporated herein by reference.

The contents of all other patents, patent applications, and references cited throughout this specification are also hereby incorporated by reference in their entireties.

### BACKGROUND OF THE INVENTION

To maximize the pharmacological benefit of any polypeptide, it is essential to have finished dosage forms that are stable, easily and reproducibly manufactured, and designed for standard routes of administration. Specifically, it is desirable to have stable, concentrated forms of bulk protein, e.g., therapeutic polypeptides which, in turn, are suitable for further manufacture into finished dosage forms of the polypeptide, which can then be administered via a desired administration route.

In both bulk polypeptide and finished dosage forms, polypeptide stability can be affected by such factors as ionic strength, pH, temperature, repeated cycles of freeze/thaw and shear forces. Active polypeptide may be lost as a result of physical instabilities, including denaturation and aggregation (both soluble and insoluble aggregate formation), as well as chemical instabilities, including, for example, hydrolysis, deamidation, and oxidation, to name just a few. For a general review of stability of protein pharmaceuticals, see, for example, Manning, et al., Pharmaceutical Research 6:903-918 (1989). In addition, it is desirable to maintain stability when carrier polypeptides are not included in the formulation.

While it is widely appreciated that these possible polypeptide instabilities can occur, until a polypeptide has been studied it is impossible to predict the particular instability problems that a particular protein may have. Any of these instabilities can potentially result in the formation of a polypeptide by-product or derivative having lowered activity, increased toxicity, and/or increased immunogenicity. Indeed, polypeptide precipitation can lead to thrombosis, non-homogeneity of dosage form and immune reactions. Thus, the safety and efficacy of any pharmaceutical formulation of a polypeptide is directly related to its stability.

Accordingly, there continues to exist a need in the art for methods for improving protein stability during the concentration process as well as providing stability in the absence of other carrier proteins in a concentration sufficiently high for various routes of administration.

### SUMMARY OF THE INVENTION

The present invention provides formulations designed to provide stability and to maintain the biological activity of an incorporated biologically active protein, in particular an antigen-binding polypeptide, for example, an antibody or fragment thereof. The invention further provides polypeptide formulations, i.e., stabilized liquid polypeptide formulations that are resistant to the formation of undesired polypeptide by-products.

The integrity of antigen-binding polypeptides for therapeutic use is especially important because if the polypeptide forms by-products, for example, aggregates or degradation fragments during storage, bioactivity may be lost, thereby jeopardizing the therapeutic activity of the molecule per unit dose. In addition, there is an acute desire to stabilize therapeutic polypeptides intended for specialized functions, for delivery and use in certain biological indications, for example, treating neurodegenerative conditions, where a polypeptide must traverse the blood-brain-barrier (BBB) and bind a target antigen.

Exemplary antibodies that must be stabilized for such use include those antibodies suitable for binding disease targets, in particular, antigenic disease targets, for example, cancer antigens, autoimmune antigens, allergens, and pathogens.

Accordingly, the invention has several advantages which include, but are not limited to, the following:
stabilized liquid polypeptide formulations which are stabilized against the formation of polypeptide by-products by the addition of an antioxidant;
stabilized liquid polypeptide formulations suitable for use in a variety of administration routes;
methods for preparing therapeutic polypeptides for pharmaceutical use as a stabilized liquid polypeptide formulations; and
stabilized Aβ-binding polypeptide formulations suitable for use in treating neurodegenerative disease.

Accordingly, in one aspect, the invention provides a stabilized liquid polypeptide formulation designed to provide stability and to maintain the biological activity of the incorporated polypeptide. In yet another aspect, the present invention provides a formulation containing a therapeutically active antigen-binding polypeptide, and an antioxidant, for example, methionine or an analog thereof, wherein the antioxidant is in an amount sufficient to reduce the by-product formation of the polypeptide during storage of the formulation.

In one embodiment, the therapeutically active antigen-binding polypeptide component of the formulation is an antibody (e.g., IgM, IgG₁, IgG₂, IgG₂, IgG₃, IgG₄), (e.g., a human IgM, IgG₁, IgG₂, IgG₂, IgG₃, IgG₄ isotype antibody) an antibody Fv fragment, an antibody Fab fragment, an antibody Fab′(2) fragment, an antibody Fd fragment, a single-chain antibody (scFv), a single domain antibody fragment (Dab), a beta-pleated sheet polypeptide comprising at least one antibody complementarity determining region (CDR), or a non-globular polypeptide comprising at least one antibody complementarity determining region (CDR).

In a particular embodiment, the liquid polypeptide formulations are stabilized against the formation of undesired by-products such as high molecular weight polypeptide aggregates, low molecular weight polypeptide degradation products, or mixtures thereof.

In a related embodiment, wherein the therapeutic antigen-binding polypeptide is an antibody, typical high molecular weight aggregates are, for example, antibody:antibody complexes, antibody:antibody fragment complexes, antibody fragment:antibody fragment complexes, or mixtures thereof. In general, high molecular weight complexes or by-products have a molecular weight greater than a monomer of the antigen-binding polypeptide, for example, in the case of an IgG antibody, greater than about 150 kD.

In another related embodiment, when the therapeutic polypeptide is an antibody, typical low molecular weight polypeptide degradation products are, for example, complexes consisting of an antibody light chain, an antibody heavy chain, an antibody light chain and heavy chain complex, or mixtures thereof. In general, low molecular weight complexes or by-products have a molecular weight less than that of a monomer of the antigen-binding polypeptide, for example, in the case of an IgG antibody, less than about 150 kD.

In one aspect, the invention provides a stabilized formulation of a therapeutically active antigen-binding polypeptide (e.g., an antibody or antigen-binding fragment thereof), methionine, where the methionine is present as an antioxidant in an amount sufficient to inhibit the formation of undesired by-products, a tonicity agent (e.g., mannitol), where the tonicity agent is present in an amount sufficient to render the formulation suitable for administration, for example, intravenous infusion, and an amino acid (e.g., histidine) or derivative thereof, where the amino acid or derivative thereof is present in an amount sufficient to maintain a physiologically suitable pH.

In one aspect, the invention provides a stabilized formulation of a therapeutically active antigen-binding polypeptide (e.g., an antibody or antigen-binding fragment) thereof, methionine, where the methionine is present as an antioxidant in an amount sufficient to inhibit the formation of undesired by-products, a tonicity agent (e.g., mannitol), where the tonicity agent is present in an amount sufficient to render the formulation suitable for intravenous infusion, and an amino acid (e.g., histidine) or derivative thereof, where the amino acid or derivative thereof is present in an amount sufficient to maintain a physiologically suitable pH.

In another aspect, the present invention provides a formulation including a therapeutically active antigen-binding polypeptide (e.g., an antibody or antigen-binding fragment thereof), mannitol and histidine. In another aspect, the invention provides a stabilized formulation including a therapeutically active antigen-binding polypeptide (e.g., an antibody or antigen-binding fragment thereof), methionine, mannitol, and histidine.

In certain embodiments, the therapeutically active antigen-binding polypeptide is an antibody (or portion or fragment thereof) that binds to an antigen selected from the an antigen class that includes, for example, cancer antigens, autoimmune antigens, allergens, and pathogens.

In certain embodiments, the therapeutically active antigen-binding polypeptide is an Aβ binding polypeptide, for example, an anti Aβ antibody (or portion or fragment thereof). In some formulations, at least one Aβ binding polypeptide is an anti Aβ antibody, for example, that specifically binds to epitope within residues 1-7, 1-5, 3-7, 3-6, 13-28, 15-24, 16-24, 16-21, 19-22, 33-40, 33-42 of Aβ, or Fab, Fab′(2) or Fv fragment thereof. Exemplary anti Aβ antibodies specifically bind to an epitope within residues 1-10 of Aβ, such as, for example, within residues 1-7, 1-5, 3-7, or 3-6 of Aβ. Other exemplary anti Aβ antibodies specifically bind to an epitope within residues 13-28 of Aβ, such as, for example, within residues 16-21 or 19-22 of Aβ. Yet other exemplary anti Aβ antibodies specifically bind to a C terminal epitope of Aβ such as, for example, 33-40 or 33-42 of Aβ. In one embodiment, the Aβ antibody is a humanized antibody, for example, a humanized 3D6 antibody, a humanized 10D5 antibody, a humanized 12B4 antibody, a humanized 15C11 antibody, or a humanized 12A11 antibody.

The therapeutically active antigen-binding polypeptide (e.g., antibody or antigen-binding fragment thereof) may be present from about 0.1 mg/ml to about 200 mg/ml (e.g., at about 20 mg/ml or 30 mg/ml). The isotype of the antibody can be IgM, IgG1, IgG2, IgG3, IgG4 or any other pharmaceutically acceptable isotype. In preferred formulations, the isotype is human IgG1 or human IgG4. In some liquid formulations, the concentration of the anti Aβ antibody is about 0.1 mg/ml to about 60 mg/ml, about 40 mg/ml to about 60 mg/ml, about 50 mg/ml, about 30 mg/ml, about 17 mg/ml to about 23 mg/ml, about 20 mg/ml, about 17 mg/ml, about 10 mg/ml, about 5 mg/ml, about 2 mg/ml, or about 1 mg/ml, preferably about 17 mg/ml to about 23 mg/ml

In certain embodiments, the mannitol is present in amount sufficient to maintain isotonicity of the formulation. Mannitol can be present from about 2% w/v to about 6% w/v (e.g., at about 4% w/v). In various embodiments of the preceding aspects, the histidine may be present in an amount sufficient to maintain a physiologically suitable pH. Histidine (e.g., L-histidine) may be present from about 0.1 mM to about 25 mM (e.g., at about 10 mM).

In other embodiments, the formulation may further include an anti-oxidant such as methionine. The methionine may be present at about 0.1 mM to about 25 mM (e.g., at about 10 mM). In another embodiment, the formulation may include a stabilizer such as polysorbate 80. The polysorbate 80 may be present from about 0.001% w/v to about 0.01% w/v (e.g., at about 0.005% w/v). In certain embodiments, the formulation has a pH of about 5 to about 7 (e.g., about 6).

In certain embodiments, the formulation may be stable to freezing. Additionally, the formulation may be suitable for administering parenterally, intravenously, intramuscularly, subcutaneously, intracranially, or epidurally. In various embodiments, the formulation may be suitable for targeted delivery to the brain or the spinal fluid of a subject. In other embodiments, the formulation may be substantially free of preservatives. The formulation may be stable for at least about 12 months, at least about 18 months, at least about 24 months, or at least about 30 months. In various embodiments, the formulation is stable at about −80° C. to about 40° C., at about 0° C. to about 25° C., or at about 2° C. to about 8° C. Some formulations are stable for at least about 12 months, at least about 18 months, at least about 24 months, or at least about 30 months. Some formulations are stable at about −80° C. to about 40° C., at about 0° C. to about 25° C., at about 0° C. to about 10° C., preferably at about −80° C. to about −50° C. or at about 2° C. to about 8° C. Some formulations are stable for at least about 12 months at a temperature of above freezing to about 10° C. and has a pH of about 5.5 to about 6.5.

In a particular aspect, the present invention provides a formulation suitable for intravenous administration including about 20 mg/mL of therapeutically active antigen-binding polypeptide (e.g., antibody or antigen-binding fragment thereof), about 10 mM L-histidine, about 10 mM methionine, about 4% mannitol and having a pH of about 6. In another aspect, the present invention provides a formulation suitable for intravenous administration including about 20 mg/mL therapeutically active antigen-binding polypeptide (e.g., antibody or antigen-binding fragment thereof, about 10 mM L-histidine, about 10 mM methionine, about 4% mannitol, about 0.01% polysorbate 80, and having a pH of about 6. In another aspect, the present invention provides a formulation suitable for intravenous administration including about 20 mg/mL therapeutically active antigen-binding polypeptide (e.g., antibody or antigen-binding fragment thereof), about 10 mM L-histidine, about 10 mM methionine, about 4% mannitol, about 0.005% polysorbate 80, and having a pH of about 6.

Some formulations are stable for at least about 12 months at a temperature of above freezing to about 10° C. and has a pH of about 5.5 to about 6.5. Such formulation includes at least one therapeutically active antigen-binding polypeptide (e.g., antibody or antigen-binding fragment thereof) at a concentration of about 1 mg/ml to about 30 mg/ml, mannitol at a concentration of about 4% w/v or NaCl at a concentration of about 150 mM, histidine or succinate at a concentration of about 5 mM to about 10 mM, and 10 mM methionine. One such formulation has a pH of about 6.0, about 1 mg/ml therapeutically active antigen-binding polypeptide (e.g., antibody or antigen-binding fragment thereof) about 10 mM histidine and about 4% w/v mannitol. Other formulations are stable for at least about 24 months at a temperature of about 2° C. to 8° C., and include polysorbate 80 at a concentration of about 0.001% w/v to about 0.01% w/v. Some of such formulations have a pH of about 6.0 to about 6.5 and include about 10 mM histidine, about 4% w/v mannitol and about 1 mg/ml, about 2 mg/ml or about 5 mg/ml therapeutically active antigen-binding polypeptide (e.g., antibody or antigen-binding fragment thereof). Other such formulations include about 10 mM histidine, about 4% w/v mannitol, about 0.005% w/v polysorbate 80 and about 10 mg/ml, about 20 mg/ml or 30 mg/ml therapeutically active antigen-binding polypeptide (e.g., antibody or antigen-binding fragment thereof), preferably at a pH of about 6.0 to about 6.2.

A preferred formulation is stable for at least about 24 months at a temperature of about 2° C. to about 8° C., has a pH of about 5.5 to about 6.5, and includes about 2 mg/ml to about 23 mg/ml, preferably about 17 mg/ml to about 23 mg/ml, of a humanized 3D6 antibody, about 10 mM histidine and about 10 mM methionine. Preferably, the formulation further includes about 4% w/v mannitol. The formulation preferably includes polysorbate 80 at a concentration of about 0.001% w/v to about 0.01% w/v, more preferably about 0.005% w/v polysorbate 80. In such formulations, the humanized 3D6 antibody can be present at a concentration of about 20 mg/ml to about 23 mg/ml.

Another formulation is stable for at least about 24 months at a temperature of about 2° C. to about 8° C., has a pH of about 5.5 to about 6.5, and includes about 2 mg/ml to about 23 mg/ml of therapeutically active antigen-binding polypeptide (e.g., antibody or antigen-binding fragment thereof), about 10 mM succinate, about 10 mM methionine, about 4% w/v mannitol and about 0.005% w/v polysorbate 80. In some of such formulations, the therapeutically active antigen-binding polypeptide (e.g., antibody or antigen-binding fragment thereof) concentration is present at a concentration of about 17 mg/ml to about 23 mg/ml.

The invention also provides a formulation that is stable when thawed from about −50° C. to about −80° C., has a pH of about 6.0 and includes about 40 to about 60 mg/ml of therapeutically active antigen-binding polypeptide (e.g., antibody or antigen-binding fragment thereof), about 1.0 mg/ml to about 2.0 mg/ml histidine, about 1.0 mg/ml to 2.0 mg/ml methionine and about 0.05 mg/ml polysorbate 80. Preferably, mannitol is excluded.

The present invention also provides a liquid formulation including therapeutically active antigen-binding polypeptide (e.g., antibody or antigen-binding fragment thereof), mannitol and histidine. In some of such formulations, the therapeutically active antigen-binding polypeptide (e.g., antibody or antigen-binding fragment thereof) is present from about 1 mg/ml to about 30 mg/ml. Preferably, the mannitol is present in an amount sufficient to maintain isotonicity of the formulation. Preferably, the histidine is present in an amount sufficient to maintain a physiologically suitable pH. One such formulation includes about 20 mg/mL therapeutically active antigen-binding polypeptide (e.g., antibody or antigen-binding fragment thereof), about 10 mM L-histidine, about 10 mM methionine, about 4% mannitol and has a pH of about 6. Another such formulation includes about 30 mg/mL therapeutically active antigen-binding polypeptide (e.g., antibody or antigen-binding fragment thereof), about 10 mM succinate, about 10 mM methionine, about 6% mannitol and has a pH of about 6.2. Yet another such formulation includes about 20 mg/mL therapeutically active antigen-binding polypeptide (e.g., antibody or antigen-binding fragment thereof), about 10 mM L-histidine, about 10 mM methionine, about 4% mannitol, about 0.005% polysorbate 80, and has a pH of about 6. Another such formulation includes about 10 mg/mL therapeutically active antigen-binding polypeptide (e.g., antibody or antigen-binding fragment thereof), about 10 mM succinate, about 10 mM methionine, about 10% mannitol, about 0.005% polysorbate 80, and has a pH of about 6.5.

Still another such formulation includes about 5 mg/mL to about 20 mg/mL therapeutically active antigen-binding polypeptide (e.g., antibody or antigen-binding fragment thereof), about 5 mM to about 10 mM L-histidine, about 10 mM methionine, about 4% mannitol, about 0.005% polysorbate 80, and has a pH of about 6.0 to about 6.5. Yet another such formulation includes about 5 mg/mL to about 20 mg/mL therapeutically active antigen-binding polypeptide (e.g., antibody or antigen-binding fragment thereof), about 5 mM to about 10 mM L-histidine, about 10 mM methionine, about 150 mM NaCl, about 0.005% polysorbate 80, and has a pH of about 6.0 to about 6.5.

The present invention also provides a formulation suitable for intravenous administration that includes about 20 mg/mL of therapeutically active antigen-binding polypeptide (e.g., antibody or antigen-binding fragment thereof), about 10 mM L-histidine, about 10 mM methionine, about 4% mannitol and has a pH of about 6. Preferably, such formulation includes about 0.005% polysorbate 80.

The invention provides a method for increasing the stability of an antigen-binding polypeptide, for example, an antibody, in a liquid pharmaceutical formulation, where the polypeptide would otherwise exhibit by-product formation during storage in a liquid formulation. Accordingly, the method comprises incorporating into the formulation an anti-oxidant, for example, methionine or an analog thereof, in an amount sufficient to reduce the amount of by-product formation.

The present invention also provides a method for maintaining the stability of a therapeutically active antigen-binding polypeptide (e.g., antibody or antigen-binding fragment thereof) formulation to be stored at a temperature of about −50° C. to about −80° C. followed by storage at a temperature of about 2° C. to about 8° C., comprising (i) combining about 40 mg/ml to about 60 mg/ml therapeutically active antigen-binding polypeptide (e.g., antibody or antigen-binding fragment thereof), about 1 mg/ml to about 2 mg/ml L-histidine, about 1 mg/ml to about 2 mg/ml methionine and about 0.05 mg/ml polysorbate 80; (ii) adjusting the pH to about 6.0; (iii) filtering into a cryovessel and freezing; (iv) thawing; (v) adding mannitol or NaCl and diluent in amounts sufficient to result in a final concentration of about 4% mannitol or about 150 mM NaCl, about 2 mg/ml to about 20 mg/ml therapeutically active antigen-binding polypeptide (e.g., antibody or antigen-binding fragment thereof); about 5 mM to about 10 mM histidine; about 10 mM methionine and about 0.005% polysorbate 80; (vi) filtering; (vii) transferring to a glass vial and sealing; and (viii) storing at a temperature of about 2° C. to about 8° C.

The present invention also provides a kit including a container with a formulation described herein and instructions for use.

The present invention also provides a pharmaceutical unit dosage form, including a formulation of about 10 mg to about 250 mg of therapeutically active antigen-binding polypeptide (e.g., antibody or antigen-binding fragment thereof), about 4% mannitol or about 150 mM NaCl, about 5 mM to about 10 mM histidine or succinate, and about 10 mM methionine. Some of such pharmaceutical unit dosage forms include about 0.001% to about 0.1% of polysorbate 80. Some of such pharmaceutical unit dosage forms include about 40 mg to about 60 mg, about 60 mg to about 80 mg, about 80 mg to about 120 mg, about 120 mg to about 160 mg, or about 160 mg to about 240 mg of the therapeutically active antigen-binding polypeptide (e.g., antibody or antigen-binding fragment thereof). Some of such formulations can be maintained in a glass vial at a temperature of about 2° C. to about 8° C. prior to administration to a patient.

In addition, the present invention provides a therapeutic product including a glass vial with a formulation including about 10 mg to about 250 mg of therapeutically active antigen-binding polypeptide (e.g., antibody or antigen-binding fragment thereof), about 4% mannitol or about 150 mM NaCl, about 5 mM to about 10 mM histidine, and about 10 mM methionine. Some of such therapeutic products further include a labeling for use including instructions to use the appropriate volume necessary to achieve a dose of about 0.15 mg/kg to about 5 mg/kg in a patient. Typically, the vial is a 1 mL, a 2 mL, a 5 mL, a 10 mL, a 25 mL or a 50 mL vial. The dose of some of such therapeutic products is about 0.5 mg/kg to about 3 mg/kg, preferably about 1 mg/kg to about 2 mg/kg. In some such therapeutic products, the therapeutically active antigen-binding polypeptide (e.g., antibody or antigen-binding fragment thereof) concentration is about 10 mg/ml to about 60 mg/ml, preferably about 20 mg/ml. The therapeutic product preferably includes about 0.005% polysorbate 80. The formulation of some such therapeutic products is for subcutaneous administration or intravenous administration.

In another aspect, the invention provides a method for increasing the stability of an antigen-binding polypeptide, for example, an antibody, in a liquid pharmaceutical formulation, where the polypeptide would otherwise exhibit by-product formation during storage in a liquid formulation. Accordingly, the method comprises incorporating into the formulation an anti-oxidant, for example, methionine or an analog thereof, in an amount sufficient to reduce the amount of by-product formation.

In yet another aspect, the present invention provides a kit including a container with a formulation described herein and instructions for use.

Other features and advantages of the invention will be apparent from the following detailed description and claims.

### BRIEF DESCRIPTION OF THE DRAWINGS

FIG. 1 depicts a schematic representation of the predicted structure of an IgG antibody and approximate positions of intra- and inter-chain disulfide bonds, glycosylation sites (hexagonal symbol), complementarity determining regions (CDRs), framework regions (shaded), and constant regions.

FIG. 2 identifies the complete amino acid sequences of the humanized 3D6 version 2 (hu3D6.v2) anti Aβ antibody light and heavy chains, SEQ ID NO:1 and SEQ ID NO:2, respectively. Light chain complementarity determining regions (CDR), i.e., CDR1, CDR2, and CDR3 are, respectively, at residue positions 24-39, 55-61, and 94-102 (upper panel). Heavy chain complementarity determining regions (CDR), i.e., CDR1, CDR2, and CDR3 are, respectively, at residue positions 40-44, 50-65, and 99-108 (lower panel). Predicted intramolecular disulfide bonds are illustrated by connections of the cysteine residues involved. Cysteines expected to form intermolecular disulfide bonds are underlined and the connectivity indicated. The N-linked glycosylation consensus site of the antibody heavy chain is indicated in bold italics at residue positions 299-301 (lower panel). The predicted heavy chain C-terminal lysine is shown in parenthesis.

FIG. 3 graphically depicts the shelf life predictions for antibody formulations (with and without polysorbate 80 (PS80)) made in accordance with the present invention and stored at 5° C.

FIG. 4 graphically depicts the shelf life predictions for antibody formulations (with and without PS80) made in accordance with the present invention and stored at 25° C.

FIG. 5 graphically depicts the shelf life predictions for antibody formulations (with and without PS80) made in accordance with the present invention and stored at 40° C.

FIG. 6 graphically depicts the degradation predictions of formulations with PS80 made in accordance with the present invention and stored at 5° C.

FIG. 7 graphically depicts the size exclusion chromatography (SEC) analysis of formulations with PS80 made in accordance with the present invention, stored at 5° C., and reprocessed to minimize assay variability.

FIG. 8 graphically depicts the degradation predictions of formulations without PS80 made in accordance with the present invention and stored at 5° C.

FIG. 9 depicts a chromatogram which indicates that the presence of PS80 shifts the by-products found within the stabilized polypeptide formulation from a high molecular weight species to a low molecular weight species without changing the monomer antibody profile.

FIG. 10 graphically depicts the inhibition of the formation of undesired by-products in a polypeptide formulation comprising IgG₄, in particular, high molecular weight polypeptide aggregates, upon the addition of an antioxidant such as free methionine.

FIG. 11 graphically depicts the inhibition of the formation of undesired by-products in a polypeptide formulation comprising IgG₂, in particular, high molecular weight polypeptide aggregates, upon the addition of an antioxidant such as free methionine.

### DETAILED DESCRIPTION OF THE INVENTION

In order to provide a clear understanding of the specification and claims, the following definitions are conveniently provided below.

As used herein, the term “antigen-binding polypeptide” includes polypeptides capable of specifically binding to a target molecule, for example, an antigen, for example, an Aβ peptide(s) or to epitope(s) within said Aβ peptides. Typically, antigen-binding polypeptides comprise at least a functional portion of an immunoglobulin or immunoglobulin-like domain (e.g., a receptor) that comprises one or more variability regions or complementarity determining regions (CDRs) which impart a specific binding characteristic to the polypeptide. Preferred antigen-binding polypeptides include antibodies, for example, IgM, IgG1, IgG2, IgG3, or IgG4.

The term “antibody” includes monoclonal antibodies (including full length monoclonal antibodies), polyclonal antibodies, multispecific antibodies (e.g., bispecific antibodies), chimeric antibodies, CDR-grafted antibodies, humanized antibodies, human antibodies, and single chain antibodies (scFvs). The term “single-chain antibody” refers to a protein having a two-polypeptide chain structure consisting of a heavy and a light chain, said chains being stabilized, for example, by interchain peptide linkers, which has the ability to specifically bind antigen. The term “antibody fragment” includes F(ab′)2 fragments, Fab fragments, Fd fragments, Fv fragments, and single domain antibody fragments (DAbs).

The term “domain” refers to a globular region of a heavy or light chain polypeptide comprising an immunoglobulin fold. The immunoglobulin fold is comprised of β-pleated sheet secondary structure and includes a disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “anti Aβ antibody” includes antibodies (and fragments thereof) that are capable of binding epitopes(s) of the Aβ peptide. Anti Aβ antibodies include, for example, those antibodies described in U.S. Patent Publication No. 20030165496A1, U.S. Patent Publication No. 20040087777A1, International Patent Publication No. WO02/46237A3, and International Patent Publication No. WO04/080419A2. Other anti Aβ antibodies are described in, e.g., International Publication Nos. WO03/077858A2 and WO04/108895A2, both entitled “Humanized Antibodies that Recognize Beta Amyloid Peptide”, International Patent Publication No. WO03/016466A2, entitled “Anti-Aβ Antibodies”, International Patent Publication No. WO0162801A2, entitled “Humanized Antibodies that Sequester Amyloid Beta Peptide”, and International Patent Publication No. WO02/088306A2, entitled “Humanized Antibodies”.

The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding).

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant cross-reactivity. In exemplary embodiments, the antibody exhibits no cross-reactivity (e.g., does not cross-react with non-Aβ peptides or with remote epitopes for example, non contiguous epitopes on Aβ). “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable protein, polypeptide or peptide). For example, an antibody that specifically binds to Aβ will appreciably bind Aβ but will not significantly react with non-Aβ proteins or peptides (e.g., non-Aβ proteins or peptides included in plaques). An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

Binding fragments are produced by recombinant DNA techniques, or by enzymatic or chemical cleavage of intact immunoglobulins. Binding fragments include Fab, Fab′, F(ab′)₂, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

An “antigen” is a molecule (e.g., a protein, polypeptide, peptide or carbohydrate) containing an antigenic determinant to which an antibody specifically binds.

The term “epitope” or “antigenic determinant” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

The term “stabilized formulation” or “stabilized liquid polypeptide formulation” includes formulations in which the polypeptide therein essentially retains its physical and chemical identity and integrity upon storage. Various analytical techniques for measuring protein stability are available in the art and are described herein (reviewed in, Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993)). Stability can be measured at a selected temperature for a selected time period. For rapid testing, the formulation may be kept at a higher or “accelerated” temperature, e.g., 40° C. for 2 weeks to 1 month or more at which time stability is measured. In exemplary embodiments, the formulation is refractory to the formation of by-products of the component polypeptide, for example, high molecular weight aggregation products, low molecular weight degradation or fragmentation products, or mixtures thereof.

The term “by-product” includes undesired products, which detract, or diminish the proportion of therapeutic polypeptide in a given formulation. Typical by-products include aggregates of the therapeutic polypeptide, fragments of the therapeutic polypeptide (e.g., produced by degradation of the polypeptide by deamidation or hydrolysis), or mixtures thereof.

The term “high molecular weight polypeptide aggregates” includes aggregates of the therapeutic polypeptide, fragments of the therapeutic polypeptide (e.g., produced by degradation of the polypeptide by, for example, hydrolysis), or mixtures thereof, that then aggregate. Typically, high molecular weight aggregates are complexes which have a molecular weight which is greater than the therapeutic monomer polypeptide. In the case of an antibody, for example, an IgG antibody, such aggregates are greater than about 150 kD. However, in the case of other therapeutic polypeptides, for example, single-chain antibodies, which typically have a molecular weight of 25 kD, such aggregates would have a molecular weight greater than about 25 kD.

The term “low molecular weight polypeptide degradation product” includes, for example, fragments of the therapeutic polypeptide, for example, brought about by deamidation or hydrolysis. Typically, low molecular weight degradation products are complexes which have a molecular weight which is less than the therapeutic monomer polypeptide. In the case of an antibody, for example, an IgG antibody, such degradation products are less than about 150 kD. However, in the case of other therapeutic polypeptides, for example, single-chain antibodies, which typically have a molecular weight of 25 kD, such aggregates would have a molecular weight less than about 25 kD.

The term “administration route” includes art recognized administration routes for delivering a therapeutic polypeptide such as, for example, parenterally, intravenously, intramuscularly, subcutaneously, intracranially, or epidurally. For the administration of a therapeutic polypeptide for the treatment of a neurodegenerative disease, intravenous, epidural, or intracranial routes, may be desired.

The term “amyloidogenic disease” includes any disease associated with (or caused by) the formation or deposition of insoluble amyloid fibrils. Exemplary amyloidogenic diseases include, but are not limited to systemic amyloidosis, Alzheimer's disease, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. Accordingly, Alzheimer's disease is an example of a “disease characterized by deposits of Aβ” or a “disease associated with deposits of Aβ”, e.g., in the brain of a subject or patient.

The terms “α-amyloid protein”, “β-amyloid peptide”, “β-amyloid”, “Aβ” and “Aβ peptide” are used interchangeably herein.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, delay, relieve, alter, remedy, ameliorate, improve or affect the disease, the symptoms of disease or the predisposition toward disease.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

The term “patient” includes human and other mammalian subjects that receive either prophylactic or therapeutic treatment.

The term “dosage unit form” (or “unit dosage form”) as used herein refers to a physically discrete unit suitable as unitary dosages for the patient to be treated, each unit containing a predetermined quantity of active compound calculated to produce the desired therapeutic effect in association with the required pharmaceutical carrier, diluent, or excipient. The specification for the dosage unit forms of the invention are dictated by and directly dependent on the unique characteristics of the active compound and the particular therapeutic effect to be achieved, and the parameters known in the art of compounding such an active compound for the treatment of patients.

Actual dosage levels of the active ingredient (e.g. Aβ polypeptides) in the formulations of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The term “diluent” as used herein refers to a solution suitable for altering or achieving an exemplary or appropriate concentration or concentrations as described herein.

Overview

The present invention provides formulations for antigen binding polypeptides, in particular, antibodies, as well as portions and/or fragments thereof. In certain aspects, the invention provides stabilized liquid polypeptide formulations for therapeutic use. In particular, the invention provides for the stabilization of antigen binding polypeptides, for example, antibodies, and antigen-binding fragments thereof, for the use in treating diseases and/or disorders. In particular, the invention provides formulations that are stabilized such that the active therapeutic polypeptide is stable over an extended period of time and can be administered through a variety of administration routes. This is especially critical for those antigen binding polypeptides (e.g., antibodies) destined for use in the treatment of certain diseases and/or disorders, e.g., neurological disease or disorder. In other aspects, the invention provides a uniquely stable antibody formulation that, for example, is stable to various stresses such as freezing, lyophilization, heat and/or reconstitution. Moreover, exemplary formulations of the present invention are capable of maintaining the stability, biological activity, purity and quality of the antibody over an extended period of time and even at unfavorable temperatures (e.g., a year during which time the formulation is stored). In addition, exemplary formulations of the present invention are suitable for administration to a subject or patient (e.g., intravenous administration to a subject or patient), for example, a human having or predicted to have a neurological disease or disorder, e.g., an amyloidogenic disease involving the amyloid Aβ polypeptide.

Formulations

In one aspect, the present invention provides a formulation including a therapeutically active antigen-binding polypeptide (e.g., an antibody or antigen-binding fragment thereof), a tonicity agent (e.g., mannitol), where the tonicity agent is present in an amount sufficient to render the formulation suitable for intravenous infusion, and an amino acid (e.g., histidine) or derivative thereof, where the amino acid or derivative thereof is present in an amount sufficient to maintain a physiologically suitable pH. In an exemplary embodiment, the present invention provides a formulation including a therapeutically active antigen-binding polypeptide (e.g., an antibody or antigen-binding fragment thereof), mannitol and histidine.

In another aspect, the present invention provides a stabilized formulation including a therapeutically active antigen-binding polypeptide. Antigen-binding polypeptides suitable for stabilization in a formulation of the invention include antibodies and fragments thereof, and in particular, antibodies capable of binding a therapeutic target involved in disease or disorder. Accordingly, the therapeutic polypeptides are stabilized according to the invention to avoid the formation of by-products, typically high molecular weight aggregates, low molecule weight degradation fragments, or a mixture thereof, by the addition of an antioxidant in a sufficient amount so as to inhibit the formation of such by-products. Antioxidant agents include methionine and analogs thereof, at concentrations sufficient to obtain the desired inhibition of undesired by-products as discussed below. Optionally, the stabilized polypeptide formulations of the invention further comprise a tonicity agent (e.g., mannitol), where the tonicity agent is present in an amount sufficient to render the formulation suitable for several different routes of administration, for example, intravenous infusion, and an amino acid (e.g., histidine) or derivative thereof, where the amino acid or derivative thereof is present in an amount sufficient to maintain a physiologically suitable pH. In an exemplary embodiment, the present invention provides a formulation including a therapeutically active antigen-binding polypeptide, methionine, mannitol and histidine.

Polypeptides for Use in the Formulations of the Invention

The polypeptide to be formulated according to the invention as described herein is prepared using techniques which are well established in the art and include, for example, synthetic techniques (such as recombinant techniques and peptide synthesis or a combination of these techniques), or may be isolated from an endogenous source of the polypeptide. In certain embodiments of the invention, the polypeptide of choice is an antigen-binding polypeptide, more preferably, an antibody, and in particular, an anti-AP antibody. Techniques for the production of an antigen-binding polypeptide, and in particular, antibodies, are described below.

Antibodies

The term “antibody” as used herein refers to immunoglobulin molecules and immunologically active portions of immunoglobulin molecules, i.e., molecules that contain an antigen binding site which specifically binds (recognizes) an antigen. Examples of immunologically active portions of immunoglobulin molecules include F(ab) and F(ab′)2 fragments which can be generated by treating the antibody with an enzyme such as pepsin or produced by art-recognized recombinant engineering techniques. Embodiments of the invention are relevant for the stabilization of antibodies, for example, polyclonal and monoclonal antibodies that bind an antigen, for example a therapeutic target antigen, such as, Aβ. The term “monoclonal antibody” or “monoclonal antibody formulation”, as used herein, refers to a population of antibody molecules that contain only one species of an antigen binding site capable of recognizing and binding to a particular epitope of a target antigen, for example, an epitope(s) of Aβ. A monoclonal antibody formulation thus typically displays a single binding specificity and affinity for a particular target antigen with which it immunoreacts.

Polyclonal Antibodies

Polyclonal antibodies can be prepared as described above by immunizing a suitable subject with an immunogen. The antibody titer in the immunized subject can be monitored over time by standard techniques, such as with an enzyme linked immunosorbent assay (ELISA) using immobilized target antigen. If desired, the antibody molecules directed against the target antigen can be isolated from the mammal (e.g., from the blood) and further purified by well known techniques, such as protein A Sepharose™ chromatography to obtain the antibody, e.g., IgG, fraction. At an appropriate time after immunization, e.g., when the anti-antigen antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 255:4980-83; Yeh et al. (1976) Proc. Natl. Acad. Sci. USA 76:2927-31; and Yeh et al. (1982) Int. J. Cancer 29:269-75). For the preparation of chimeric polyclonal antibodies, see Buechler et al. U.S. Pat. No. 6,420,113.

Monoclonal Antibodies

Any of the many well known protocols used for fusing lymphocytes and immortalized cell lines can be applied for the purpose of generating a monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a target antigen, e.g., Aβ, using a standard ELISA assay.

Recombinant Antibodies

Alternative to preparing monoclonal antibody-secreting hybridomas, a monoclonal antibody can be identified and isolated by screening a recombinant combinatorial immunoglobulin library (e.g., an antibody phage display library) with a target antigen to thereby isolate immunoglobulin library members that bind the target antigen. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Chimeric and Humanized Antibodies

Additionally, recombinant antibodies, such as chimeric and humanized monoclonal antibodies, comprising both human and non-human portions, which can be made using standard recombinant DNA techniques, are within the scope of the invention.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The phrase “substantially from a human immunoglobulin or antibody” or “substantially human” means that, when aligned to a human immunoglobulin or antibody amino sequence for comparison purposes, the region shares at least 80-90%, 90-95%, or 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably at least 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The term “significant identity” means that two polypeptide sequences, when optimally aligned, such as by the programs GAP or BESTFIT using default gap weights, share at least 50-60% sequence identity, preferably at least 60-70% sequence identity, more preferably at least 70-80% sequence identity, more preferably at least 80-90% identity, even more preferably at least 90-95% sequence identity, and even more preferably at least 95% sequence identity or more (e.g., 99% sequence identity or more). The term “substantial identity” means that two polypeptide sequences, when optimally aligned, such as by the programs GAP or BESTFIT using default gap weights, share at least 80-90% sequence identity, preferably at least 90-95% sequence identity, and more preferably at least 95% sequence identity or more (e.g., 99% sequence identity or more). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

Preferably, residue positions which are not identical differ by conservative amino acid substitutions. For purposes of classifying amino acids substitutions as conservative or nonconservative, amino acids are grouped as follows: Group I (hydrophobic sidechains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Preferably, humanized immunoglobulins or antibodies bind antigen with an affinity that is within a factor of three, four, or five of that of the corresponding non-humanized antibody. For example, if the nonhumanized antibody has a binding affinity of 10⁹ M⁻¹, humanized antibodies will have a binding affinity of at least 3×10⁹ M⁻¹, 4×10⁹ M⁻¹ or 5×10⁹ M⁻¹. When describing the binding properties of an immunoglobulin or antibody chain, the chain can be described based on its ability to “direct antigen (e.g., Aβ) binding”. A chain is said to “direct antigen binding” when it confers upon an intact immunoglobulin or antibody (or antigen binding fragment thereof) a specific binding property or binding affinity. A mutation (e.g., a backmutation) is said to substantially affect the ability of a heavy or light chain to direct antigen binding if it affects (e.g., decreases) the binding affinity of an intact immunoglobulin or antibody (or antigen binding fragment thereof) comprising said chain by at least an order of magnitude compared to that of the antibody (or antigen binding fragment thereof) comprising an equivalent chain lacking said mutation. A mutation “does not substantially affect (e.g., decrease) the ability of a chain to direct antigen binding” if it affects (e.g., decreases) the binding affinity of an intact immunoglobulin or antibody (or antigen binding fragment thereof) comprising said chain by only a factor of two, three, or four of that of the antibody (or antigen binding fragment thereof) comprising an equivalent chain lacking said mutation.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human Antibodies from Transgenic Animals and Phage Display

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice results in the production of human antibodies upon antigen challenge. See, e.g., U.S. Pat. Nos. 6,150,584; 6,114,598; and 5,770,429.

Fully human antibodies can also be derived from phage-display libraries (Hoogenboom et al., J. Mol. Biol., 227:381 (1991); Marks et al., J. Mol. Biol., 222:581-597 (1991)).

Bispecific Antibodies, Antibody Fusion Polypeptides, and Single-Chain Antibodies

Bispecific antibodies (BsAbs) are antibodies that have binding specificities for at least two different epitopes. Such antibodies can be derived from full length antibodies or antibody fragments (e.g. F(ab)′2 bispecific antibodies). Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of different antibody molecules (see, WO 93/08829 and in Traunecker et al., EMBO J., 10:3655-3659 (1991)).

Bispecific antibodies also include cross-linked or “heteroconjugate” antibodies. For example, one of the antibodies in the heteroconjugate can be coupled to avidin, the other to biotin to biotin or other payload. Heteroconjugate antibodies may be made using any convenient cross-linking methods. Suitable cross-linking agents are well known in the art, and are disclosed in U.S. Pat. No. 4,676,980, along with a number of cross-linking techniques.

In yet another embodiment, the antibody can be fused, chemically or genetically, to a payload domain, such as a reactive, detectable, or functional moiety, for example, an immunotoxin to produce an antibody fusion polypeptide. Such payloads include, for example, immunotoxins, chemotherapeutics, and radioisotopes, all of which are well-known in the art.

Single chain antibodies are also suitable for stabilization according to the invention. The fragments comprise a heavy-chain variable domain (VH) connected to a light-chain variable domain (VL) with a linker, which allows each variable region to interface with each other and recreate the antigen binding pocket of the parent antibody from which the VL and VH regions are derived. See Gruber et al., J. Immunol., 152:5368 (1994).

It is understood that any of the foregoing polypeptide molecules, alone or in combination, are suitable for preparation as stabilized formulations according to the invention.

Therapeutic Antigen-Binding Polypeptides

A number of therapeutic antigen-binding polypeptides are suitable for being formulated according to the stabilizing conditions of the present invention. Typically, the antigen-binding polypeptides are antibodies or fragments thereof (see supra), that comprise an antibody variable region and/or antibody Fc region or at least a portion of an immunoglobulin, immunoglobulin superfamily protein, or receptor or receptor-like domain, that can interact with a target antigen or a molecule of the immune system, for example, an Fc receptor. For convenience, the antigen-binding polypeptides that can benefit from the methods and formulations of the present invention are discussed below according to their target antigen class. Such representative antigen-binding polypeptides bind to antigen classes that include, for example, cancer antigens, autoimmune antigens, allergens, and pathogens.

Therapeutic Antigen-Binding Polypeptides that Bind Cancer Antigens

In certain embodiments, the antigen-bind polypeptides subject to the methods and compositions of the present invention can bind a molecule specific for tumor cells for example, a tumor specific antigen. Such tumor specific antigens include, e.g., bullous pemphigoid antigen 2, prostate mucin antigen (PMA), tumor associated Thomsen-Friedenreich antigen, prostate-specific antigen (PSA), luminal epithelial antigen (LEA. 135) of breast carcinoma and bladder transitional cell carcinoma (TCC), cancer-associated serum antigen (CASA) and cancer antigen 125 (CA 125), the epithelial glycoprotein 40 (EGP40), squamous cell carcinoma antigen (SCC), cathepsin E, tyrosinase in melanoma, cell nuclear antigen (PCNA) of cerebral cavemomas, DF3/MUC1 breast cancer antigen, carcinoembryonic antigen, tumor-associated antigen CA 19-9, human melanoma antigens MART-I/Melan-A27-35 and gp100, the T and Tn pancarcinoma (CA) glycopeptide epitopes, a 35 kD tumor-associated autoantigen in papillary thyroid carcinoma, KH-1 adenocarcinoma antigen, the A60 mycobacterial antigen, heat shock proteins (HSPs), mutant oncogene products, e.g., p53, ras, and HER-2/neu.

Therapeutic Antigen-Binding Polypeptides that Bind Molecules of Inflammation and Autoimmune Disease

In certain embodiments, the antigen-binding polypeptides subject to the methods and compositions of the present invention can bind a molecule responsible for inflammation or an autoimmune disease or disorder. Such antigen-binding polypeptides can bind to molecules associated with rheumatoid arthritis, SLE, diabetes mellitus, myasthenia gravis, reactive arthritis, ankylosing spondylitis, multiple sclerosis, IBD, psoriasis, pancreatitis, and various immunodeficiencies. Other target antigens include 2-GPI, 50 kDa glycoprotein, Ku (p70/p80) autoantigen, or its 80-kd subunit protein, the nuclear autoantigens La (SS-B) and Ro (SS-A), scleroderma antigens Rpp 30, Rpp 38 or Scl-70, the centrosome autoantigen PCM-1, polymyositis-scleroderma autoantigen (PM-Scl), scleroderma (and other systemic autoimmune disease) autoantigen CENP-A, U5, a small nuclear ribonucleoprotein (snRNP), the 100-kd protein of PM-Scl autoantigen, the nucleolar U3- and Th(7-2) ribonucleoproteins, the ribosomal protein L7, the 36-kd protein from nuclear matrix antigen, insulin, proinsulin, GAD65 and GAD67, heat-shock protein 65 (hsp65), and islet-cell antigen 69 (ICA69), islet cell antigen-related protein-tyrosine phosphatase (PTP), GM2-1 ganglioside, glutamic acid decarboxylase (GAD), islet cell antigen (ICA69), Tep69, the single T cell epitope recognized by T cells from diabetes patients, ICA 512, an autoantigen of type I diabetes, an islet-cell protein tyrosine phosphatase and the 37-kDa autoantigen derived from it in type 1 diabetes (including IA-2, IA-2), the 64 kDa protein from In-111 cells or human thyroid follicular cells that is immunoprecipitated with sera from patients with islet cell surface antibodies (ICSA). In particular, rheumatoid arthritis antigens include 45 kDa DEK nuclear antigen, in particular onset juvenile rheumatoid arthritis and iridocyclitis, human cartilage glycoprotein-39, an autoantigen in rheumatoid arthritis, a 68 k autoantigen in rheumatoid arthritis, collagen, collagen type II, cartilage link protein, ezrin, radixin and moesin, mycobacterial heat shock protein 65, thyroid peroxidase and the thyroid stimulating hormone receptor, thyroid peroxidase from human Graves' thyroid tissue, a 64-kDa antigen associated with thyroid-associated ophthalmopathy, the human TSH receptor, and the 64 kDa protein from In-111 cells or human thyroid follicular cells that is immunoprecipitated with sera from patients with islet cell surface antibodies.

Therapeutic Antigen-Binding Polypeptides that Bind Allergens

In certain embodiments, the antigen-binding polypeptides subject to the methods and compositions of the present invention can bind an allergen or molecule responsible for an allergic disease or disorder. Such antigen-binding polypeptides can bind to IgE, IgE receptors, T cell receptor (TCR), cytokines, or allergens, for example, from the house dust mite, grass pollen, birch pollen, ragweed pollen, hazel pollen, cockroach, rice, olive tree pollen, fungi, mustard, bee venom, animal allergens, e.g., from horse, dog or cat, and the like. Allergens also include latex allergens.

Therapeutic Antigen-Binding Polypeptides that Bind Pathogens and Associated Toxins

In certain embodiments, the antigen-binding polypeptides subject to the methods and compositions of the present invention can bind a pathogen, for example, a bacterial, fungal, or viral pathogen, or, for example, a toxin thereof. Such antigen-binding polypeptides bind to pathogens (or toxins thereof) that include Yersinia, e.g., Yersinia pestis, the causative agent of plague, in particular the V antigen, Bacillus anthracis, the causative agent of anthrax, in particular, the anthrax protective antigen (PA) or lethal factor (LF), Staphylococcus, e.g., S. aureus and S. epidermidis, and Streptococcus and/or their associated toxins, E. coli, for example, strain O-157:H7 that causes food-borne illness; Cholera bacterium, e.g., Vibrio cholerae, or enterotoxin thereof; Helicobacter pylori, e.g., antigens CagA and VacA; Chlamydia; Neisseria gonorrhoeae; and Meisseria meningitidis; Bordetella pertussis; Brucella abortus; meningococcal antigens; pneumococcal antigens; Listeria monocytogenes; Salmonella; Shigella and Mycobacterium tuberculosis; viral pathogens, e.g., Hanta virus, flaviviruses, influenza; HIV, e.g., antigens Gag, Pol, Vif and Nef; rotavirus; herpes simplex virus-type I/II; Hepatitis A, B, C; or G; rabies; papillomavirus; Epstein-Barr virus (EBV); measles; CMV; and parasites.

Anti Aβ Antibodies

Generally, the formulations of the present invention include a variety of antibodies for treating amyloidogenic diseases, in particular, Alzheimer's Disease, by targeting Aβ peptide.

The terms “Aβ antibody”, “anti Aβ antibody” and “anti Aβ” are used interchangeably herein to refer to an antibody that binds to one or more epitopes or antigenic determinants of the human amyloid precursor protein (APP), Aβ protein, or both. Exemplary epitopes or antigenic determinants can be found within APP, but are preferably found within the Aβ peptide of APP. Multiple isoforms of APP exist, for example APP⁶⁹⁵, APP⁷⁵¹ and APP⁷⁷⁰. Amino acids within APP are assigned numbers according to the sequence of the APP⁷⁷⁰ isoform (see e.g., GenBank Accession No. P05067). Examples of specific isotypes of APP which are currently known to exist in humans are the 695 amino acid polypeptide described by Kang et. al. (1987) Nature 325:733-736 which is designated as the “normal” APP; the 751 amino acid polypeptide described by Ponte et al. (1988) Nature 331:525-527 (1988) and Tanzi et al. (1988) Nature 331:528-530; and the 770-amino acid polypeptide described by Kitaguchi et. al. (1988) Nature 331:530-532. As a result of proteolytic processing of APP by different secretase enzymes in vivo or in situ, Aβ is found in both a “short form”, 40 amino acids in length, and a “long form”, ranging from 42-43 amino acids in length. The short form, Aβ₄₀, consists of residues 672-711 of APP. The long form, e.g., Aβ₄₂ or Aβ₄₃, consists of residues 672-713 or 672-714, respectively. Part of the hydrophobic domain of APP is found at the carboxy end of Aβ, and may account for the ability of Aβ to aggregate, particularly in the case of the long form. Aβ peptide can be found in, or purified from, the body fluids of humans and other mammals, e.g. cerebrospinal fluid, including both normal individuals and individuals suffering from amyloidogenic disorders.

The terms “β-amyloid protein”, “β-amyloid peptide”, “β-amyloid”, “Aβ” and “Aβ peptide” are used interchangeably herein. Aβ peptide (e.g., Aβ39, Aβ40, Aβ41, Aβ42 and Aβ43) is a ˜4-kDa internal fragment of 39-43 amino acids of APP. Aβ40, for example, consists of residues 672-711 of APP and Aβ42 consists of residues 672-713 of APP. Aβ peptides include peptides resulting from secretase cleavage of APP and synthetic peptides having the same or essentially the same sequence as the cleavage products. Aβ peptides can be derived from a variety of sources, for example, tissues, cell lines, or body fluids (e.g. sera or cerebrospinal fluid). For example, an Aβ can be derived from APP-expressing cells such as Chinese hamster ovary (CHO) cells stably transfected with APP_{717V→F}, as described, for example, in Walsh et al., (2002), Nature, 416, pp 535-539. An Aβ preparation can be derived from tissue sources using methods previously described (see, e.g., Johnson-Wood et al., (1997), Proc. Natl. Acad. Sci. USA 94:1550). Alternatively, Aβ peptides can be synthesized using methods which are well known to those in the art. See, for example, Fields et al., Synthetic Peptides: A User's Guide, ed. Grant, W.H. Freeman & Co., New York, N.Y., 1992, p 77). Hence, peptides can be synthesized using the automated Merrifield techniques of solid phase synthesis with the α-amino group protected by either t-Boc or F-moc chemistry using side chain protected amino acids on, for example, an Applied Biosystems Peptide Synthesizer Model 430A or 431. Longer peptide antigens can be synthesized using well known recombinant DNA techniques. For example, a polynucleotide encoding the peptide or fusion peptide can be synthesized or molecularly cloned and inserted in a suitable expression vector for the transfection and heterologous expression by a suitable host cell. Aβ peptide also refers to related Aβ sequences that results from mutations in the Aβ region of the normal gene.

The term “epitope” or “antigenic determinant” refers to a site on an antigen to which an immunoglobulin or antibody (or antigen binding fragment thereof) specifically binds. Exemplary epitopes or antigenic determinants to which an Aβ antibody binds can be found within the human amyloid precursor protein (APP), but are preferably found within the Aβ peptide of APP. Exemplary epitopes or antigenic determinants within Aβ are located within the N-terminus, central region, or C-terminus of Aβ. An “N-terminal epitope”, is an epitope or antigenic determinant located within the N-terminus of the Aβ peptide. Exemplary N-terminal epitopes include residues within amino acids 1-10 or 1-12 of Aβ, preferably from residues 1-3, 1-4, 1-5, 1-6, 1-7, 2-6, 2-7, 3-6, or 3-7 of Aβ42. Other exemplary N-terminal epitopes start at residues 1-3 and end at residues 7-11 of Aβ. Additional exemplary N-terminal epitopes include residues 2-4, 5, 6, 7 or 8 of Aβ, residues 3-5, 6, 7, 8 or 9 of Aβ, or residues 4-7, 8, 9 or 10 of Aβ42. “Central” epitopes are epitopes or antigenic determinants comprising residues located within the central or mid-portion of the Aβ peptide. Exemplary central epitopes include residues within amino acids 13-28 of Aβ, preferably from residues 14-27, 15-26, 16-25, 17-24, 18-23, or 19-22 of Aβ. Other exemplary central epitopes include residues within amino acids 16-24, 16-23, 16-22, 16-21, 18-21, 19-21, 19-22, 19-23, or 19-24 of Aβ. “C-terminal” epitopes or antigenic determinants are located within the C-terminus of the Aβ peptide and include residues within amino acids 33-40, 33-41, or 33-42 of Aβ. Additional exemplary C-terminal epitopes or antigenic determinants include residues 33-40 of Aβ.

When an antibody is said to bind to an epitope within specified residues, such as Aβ 3-7, what is meant is that the antibody specifically binds to a polypeptide containing the specified residues (i.e., Aβ 3-7 in this an example). Such an antibody does not necessarily contact every residue within Aβ 3-7. Nor does every single amino acid substitution or deletion within Aβ 3-7 necessarily significantly affect binding affinity. In various embodiments, an Aβ antibody is end-specific. As used herein, the term “end-specific” refers to an antibody which specifically binds to the N-terminal or C-terminal residues of an Aβ peptide but that does not recognize the same residues when present in a longer Aβ species comprising the residues or in APP. In various embodiments, an Aβ antibody is “C-terminus-specific.” As used herein, the term “C terminus-specific” means that the antibody specifically recognizes a free C-terminus of an Aβ peptide. Examples of C terminus-specific Aβ antibodies include those that: recognize an Aβ peptide ending at residue 40 but do not recognize an Aβ peptide ending at residue 41, 42, and/or 43; recognize an Aβ peptide ending at residue 42 but do not recognize an Aβ peptide ending at residue 40, 41, and/or 43; etc.

In one embodiment, the Aβ antibody may be a 3D6 antibody or variant thereof, or a 10D5 antibody or variant thereof, both of which are described in U.S. Patent Publication No. 20030165496A1, U.S. Patent Publication No. 20040087777A1, International Patent Publication No. WO02/46237A3 and International Patent Publication No. WO04/080419A2. Description of 3D6 and 10D5 antibodies can also be found, for example, in International Patent Publication No. WO02/088306A2 and International Patent Publication No. WO02/088307A2. Additional 3D6 antibodies are described in U.S. patent application Ser. No. 11/303,478 and International Application No. PCT/US05/45614. 3D6 is a monoclonal antibody (mAb) that specifically binds to an N-terminal epitope located in the human β-amyloid peptide, specifically, residues 1-5. By comparison, 10D5 is a mAb that specifically binds to an N-terminal epitope located in the human β-amyloid peptide, specifically, residues 3-6. In another embodiment, the antibody may be a 12B4 antibody or variant thereof, as described in U.S. Patent Publication No. 20040082762A1 and International Patent Publication No. WO03/077858A2. 12B4 is a mAb that specifically binds to an N-terminal epitope located in the human β-amyloid peptide, specifically, residues 3-7. In yet another embodiment, the antibody may be a 12A11 antibody or a variant thereof, as described in U.S. Patent Publication No. 20050118651A1 and International Patent Publication No. WO04/10885A2. 12A11 is a mAb that specifically binds to an N-terminal epitope located in the human β-amyloid peptide, specifically, residues 3-7. In yet another embodiment, the antibody may be a 15C11 antibody or variant thereof, as described in U.S. patent application Ser. No. 11/304,986 and International Patent Application No. PCT/US05/45515 entitled “Humanized Antibodies that Recognize Beta Amyloid Peptide.” 15C11 is a mAb that specifically binds to a central epitope located in the human β-amyloid peptide, specifically, residues 19-22. In yet another embodiment, the antibody may be a 266 antibody as described in U.S. Patent Publication No. 20050249725A1, and International Patent Publication No. WO01/62801A2. Antibodies designed to specifically bind to C-terminal epitopes located in human β-amyloid peptide, for use in the present invention include, but are not limited to, 369.2B, as described in U.S. Pat. No. 5,786,160.

Antibodies for use in the present invention may be recombinantly or synthetically produced. For example, the antibody may be produced by a recombinant Chinese hamster ovary (CHO) cell culture process. In addition, antibodies with minor modifications that retain the primary functional property of binding Aβ peptide are contemplated by the present invention. In a particular embodiment, the antibody is a humanized anti Aβ peptide 3D6 antibody that selectively binds Aβ peptide. More specifically, the humanized anti Aβ peptide 3D6 antibody is designed to specifically bind to an NH₂-terminal epitope located in the human β-amyloid 1-40 or 1-42 peptide found in plaque deposits in the brain (e.g., in patients suffering from Alzheimer's disease).

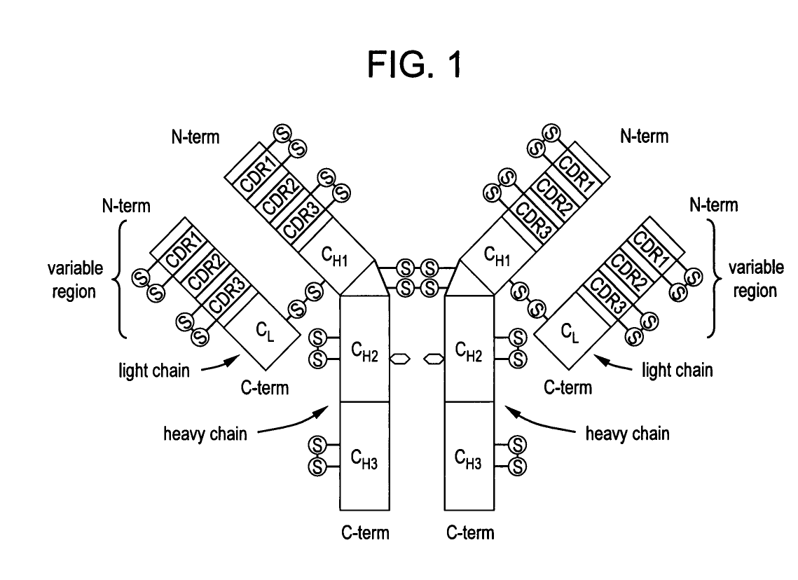

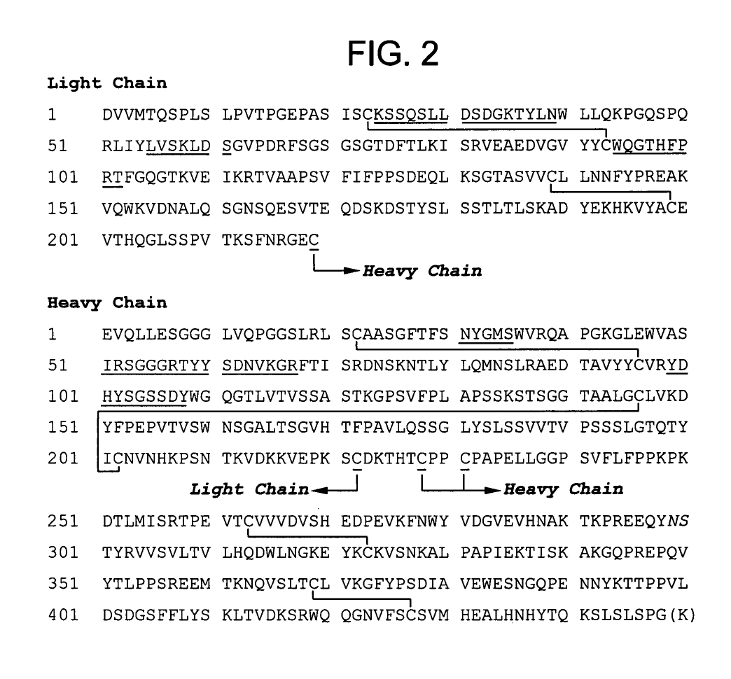

FIG. 1 provides a schematic representation of the predicted structure of an exemplary humanized anti Aβ peptide 3D6 antibody termed h3D6v2. The complete amino acid sequences of the h3D6v2 light and heavy chains predicted from the DNA sequences of the corresponding expression vectors are shown in FIG. 2 (where the residues are numbered starting with the NH₂-terminus of light and heavy chains as residue number 1). The last amino acid residue encoded by the heavy chain DNA sequence, Lys⁴⁴⁹, has not been observed in the mature, secreted form of h3D6v2 and, without wishing to be bound to any particular theory, is presumably removed during intracellular processing by CHO cellular proteases. Therefore, the COOH-terminus of the h3D6v2 heavy chain is optionally Gly⁴⁴⁸. COOH-terminal lysine processing has been observed in recombinant and plasma-derived antibodies and does not appear to impact their function (Harris (1995) J. Chromatogr. A. 705:129-134). Purified h3D6v2 is post-translationally modified by addition of N-linked glycans to the Fc portion of heavy chain, which is known to contain a single N-glycosylation consensus site. The N-glycosylation site displays three major complex biantennary neutral oligosaccharide structures commonly observed at the analogous N-glycosylation site of mammalian IgG proteins.

Another exemplary humanized anti Aβ peptide antibody is humanized 3D6 version 1 (hu3D6v1) having the sequence set forth in FIG. 2 but for a D→Y substitution at position 1 of the light chain.

In various embodiments of the present invention, the anti Aβ antibody (e.g., a humanized anti Aβ peptide 3D6 antibody) is present from about 0.1 mg/ml to about 100 mg/ml, from about 0.1 mg/ml to about 75 mg/ml, from about 0.1 mg/ml to about 50 mg/ml, from about 0.1 mg/ml to about 60 mg/ml, from about 0.1 mg/ml to about 40 mg/ml, from about 0.1 mg/ml to about 30 mg/ml, from about 10 mg/ml to about 20 mg/ml, from about 20 mg/ml to 30 mg/ml, or higher, for example, up to about 100 mg/ml, about 200 mg/ml, about 500 mg/ml, or about 1000 mg/ml or more. In various embodiments, the anti Aβ antibody is present at about 15, 16, 17, 18, 19, 20, 21, 22, 23, 24 or 25 mg/ml. In a particular embodiment, the antibody (e.g., a humanized anti Aβ peptide 3D6 antibody) is present at about 17 mg/ml. In another particular embodiment, the antibody (e.g., a humanized anti Aβ peptide 3D6 antibody) is present at about 20 mg/ml. In another particular embodiment, the antibody (e.g., a humanized anti Aβ peptide 3D6 antibody) at about 30 mg/ml. Ranges intermediate to the above recited concentrations, e.g., about 12 mg/ml to about 17 mg/ml, are also intended to be part of this invention. For example, ranges of values using a combination of any of the above recited values as upper and/or lower limits are intended to be included.

Excipients

In various embodiments, the present invention provides a formulation that may include various excipients, including, but not limited to, buffer, anti-oxidant, a tonicity agent, and a stabilizer. In addition, the formulations may contain an agent for pH adjustment (e.g., HCl) and a diluent (e.g., water). In part, the excipients serve to, in part, maintain stability and the biological activity of the antibody (e.g., by maintaining the proper conformation of the protein), and/or to maintain pH.

Buffering Agent

In various aspects of the present invention, the formulation includes a buffering agent (buffer). The buffer can serve to enhance isotonicity and chemical stability of the formulation. In addition, the buffer serves to maintain a physiologically suitable pH (e.g., a pH of about 6.0). Generally, the formulation should have a physiologically suitable pH. In various embodiments of the present invention, the formulation should have a pH of about 5 to about 7 or from about 5.5 to about 6.5. In a particular embodiment, the formulation has a pH of about 6. Ranges intermediate to the above recited pH levels, e.g., about pH 5.2 to about pH 6.3 (e.g., pH 6.2), are also intended to be part of this invention. For example, ranges of values using a combination of any of the above recited values as upper and/or lower limits are intended to be included. The pH may be adjusted as necessary by techniques known in the art. For example, HCl may be added as necessary to adjust the pH to desired levels or different forms of histidine may be added as necessary to adjust the pH to desired levels.

The buffer may include, but is not limited to, succinate (sodium or phosphate), histidine, phosphate (sodium or potassium), Tris(tris(hydroxymethyl)aminomethane), diethanolamine, citrate, other organic acids and mixtures thereof. In a particular embodiment, the buffer is histidine (e.g., L-histidine). In another particular embodiment, the buffer is succinate. In another embodiment, the formulation includes an amino acid such as histidine that is present in an amount sufficient to maintain the formulation at a physiologically suitable pH. Histidine is an exemplary amino acid having buffering capabilities in the physiological pH range. Histidine derives its buffering capabilities from its imidazole group. In one exemplary embodiment, the buffer is L-histidine (base) (e.g. C₆H₉N₃O₂, FW: 155.15). In another embodiment, the buffer is L-histidine monochloride monohydrate (e.g. C₆H₉N₃O₂.HCl.H₂O, FW: 209.63). In another exemplary embodiment, the buffer is a mixture of L-histidine (base) and L-histidine monochloride monohydrate.

In one embodiment, the buffer (e.g., L-histidine or succinate) is present from about 0.1 mM to about 50 mM, from about 0.1 mM to about 40 mM, from about 0.1 mM to about 25 mM, from about 0.1 mM to about 30 mM, from about 0.1 mM to about 20 mM, or from about 5 mM to about 15 mM, preferably about 5 mM or 10 mM. In various embodiments, the buffer may be present at about 5 mM, 6 mM, 7 mM, 8 mM, 9 mM, 10 mM, 11 mM, 12 mM, 13 mM, 14 mM, or 15 mM. In a particular embodiment, the buffer is present at about 10 mM. Ranges intermediate to the above recited concentrations, e.g., about 12 mM to about 17 mM, are also intended to be part of this invention. For example, ranges of values using a combination of any of the above recited values as upper and/or lower limits are intended to be included. In certain embodiments, the buffer is present in an amount sufficient to maintain a physiologically suitable pH.

Tonicity Agent

In various aspects of the present invention, the formulation includes a tonicity agent. In part, the tonicity agent contributes to maintaining the isotonicity of the formulation, and to maintaining protein levels. In part, the tonicity agent contributes to preserving the level, ratio, or proportion of the therapeutically active polypeptide present in the formulation. As used herein, the term “tonicity” refers to the behavior of biologic components in a fluid enviornment or solution. Isotonic solutions possess the same osmotic pressure as blood plasma, and so can be intravenously infused into a subject without changing the osmotic pressure of the subject's blood plasma. Indeed, in one embodiment according to the invention, tonicity agent is present in an amount sufficient to render the formulation suitable for intravenous infusion. Often, the tonicity agent serves as a bulking agent as well. As such, the agent may allow the protein to overcome various stresses such as freezing and shear.

The tonicity agent may include, but is not limited to, CaCl₂, NaCl, MgCl₂, lactose, sorbitol, sucrose, mannitol, trehalose, raffinose, polyethylene glycol, hydroxyethyl starch, glycine and mixtures thereof. In a particular embodiment, the tonicity agent is mannitol (e.g., D-mannitol, e.g., C₆H₁₄O₆, FW: 182.17).

In one embodiment, the tonicity agent (e.g., mannitol) is present at about 2% to about 6% w/v, or about 3% to about 5% w/v. In another embodiment, the tonicity agent is present at about 3.5% to about 4.5% w/v. In another embodiment, the tonicity agent is percent at about 20 mg/ml to about 60 mg/ml, at about 30 mg/ml to about 50 mg/ml, or at about 35 mg/ml to about 45 mg/ml. In a particular embodiment, the tonicity agent is present at about 4% w/v or at about 40 mg/ml. In another particular embodiment, the tonicity agent is present at about 6% w/v. In yet another particular embodiment, the tonicity agent is present at about 10% w/v.

Ranges intermediate to the above recited concentrations, e.g., about 3.2% to about 4.3% w/v or about 32 to about 43 mg/ml, are also intended to be part of this invention. For example, ranges of values using a combination of any of the above recited values as upper and/or lower limits are intended to be included. The tonicity agent should be present in a sufficient amount so as to maintain tonicity of the formulation.

Anti-Oxidant

In various aspects of the present invention, the formulation includes an anti-oxidant so as to, in part, preserve the formulation (e.g., by preventing oxidation).

The anti-oxidant may include, but is not limited to, GLA (gamma-linolenic acid)-lipoic acid, DHA (docosahexaenoic acid)-lipoic acid, GLA-tocopherol, di-GLA-3,3′-thiodipropionic acid and in general any of, for example, GLA, DGLA (dihomo-gamma-linolenic acid), AA (arachidonic acid), SA (salicylic acid), EPA (eicosapentaenoic acid) or DHA (docosahexaenoic acid) with any natural or synthetic anti-oxidant with which they can be chemically linked. These include phenolic anti-oxidants (e.g., eugenol, camosic acid, caffeic acid, BHT (butylated hydroxyanisol), gallic acid, tocopherols, tocotrienols and flavenoid anti-oxidants (such as myricetin and fisetin)), polyenes (e.g., retinoic acid), unsaturated sterols (e.g., Δ⁵-avenosterol), organosulfur compounds (e.g., allicin), terpenes (e.g., geraniol, abietic acid) and amino acid antioxidants (e.g., methionine, cysteine, camosine). In one embodiment, the anti-oxidant is ascorbic acid. In a particular embodiment, the anti-oxidant is methionine, or an analog thereof, e.g., selenomethionine, hydroxy methyl butanoic acid, ethionine, or trifluoromethionine.

In one embodiment, the anti-oxidant (e.g., a methionine such as L-methionine, e.g. CH₃SCH₂CH₂CH(NH₂)CO₂H, FW=149.21) is present from about 0.1 mM to about 50 mM, from about 0.1 mM to about 40 mM, from about 0.1 mM to about 30 mM, from about 0.1 mM to about 20 mM, or from about 5 mM to about 15 mM. In various embodiments, the anti-oxidant may be present at about 5 mM, 6 mM, 7 mM, 8 mM, 9 mM, 10 mM, 11 mM, 12 mM, 13 mM, 14 mM, or 15 mM. In a particular embodiment, the anti-oxidant is present at about 10 mM. In another particular embodiment, the anti-oxidant is present at about 15 mM. Ranges intermediate to the above recited concentrations, e.g., about 12 mM to about 17 mM, are also intended to be part of this invention. For example, ranges of values using a combination of any of the above recited values as upper and/or lower limits are intended to be included. In certain embodiments, the anti-oxidant should be present in a sufficient amount so as to preserve the formulation, in part, by preventing oxidation.

Stabilizer

In various aspects of the present invention, the formulation includes a stabilizer, also known as a surfactant. Stabilizers are specific chemical compounds that interact and stabilize biological molecules and/or general pharmaceutical excipients in a formulation. In certain embodiments, stabilizers may be used in conjunction with lower temperature storage. Stabilizers generally protect the protein from air/solution interface induced stresses and solution/surface induced stresses, often resulting in protein aggregation.

The stabilizer may include, but is not limited to, glycerin, polysorbates such as polysorbate 80, dicarboxylic acids, oxalic acid, succinic acid, adipic acid, fumaric acid, phthalic acids, and combinations thereof. In a particular embodiment the stabilizer is polysorbate 80.

In one embodiment, the stabilizer (e.g., polysorbate 80) is present between about 0.001% w/v to about 0.01% w/v, between about 0.001% w/v to about 0.009% w/v, or between about 0.003% w/v to about 0.007% w/v. In a particular embodiment, the stabilizer is present at about 0.005% w/v of the formulation. In another particular embodiment, the stabilizer is present at about 0.01% w/v. Ranges intermediate to the above recited concentrations, e.g., about 0.002% w/v to about 0.006% w/v, are also intended to be part of this invention. For example, ranges of values using a combination of any of the above recited values as upper and/or lower limits are intended to be included. The stabilizer should be present in a sufficient amount so as to stabilize the Aβ binding polypeptide (e.g., anti Aβ antibody).

Other pharmaceutically acceptable carriers, excipients or stabilizers such as those described in Remington's Pharmaceutical Sciences 16^{th} edition, Osol, A. Ed. (1980) may be included in the formulation provided that they do not adversely affect the desired characteristics of the formulation. In a particular embodiment, the formulation is substantially free of preservatives, although, in alternative embodiments, preservatives may be added as necessary. For example, cryoprotectants or lyoprotectants may be included, for example, should the formulation be lyophilized.

In various aspects of the present invention, the formulations optionally include some or all of the classes of excipients described above. In one aspect, the formulations of the present invention include an antigen-binding polypeptide (e.g., anti Aβ antibody, mannitol and histidine. In particular embodiments, the formulations may include an anti-oxidant such as methionine, and/or a stabilizer such as polysorbate 80. In certain embodiments, the formulations have a pH of about 6. In another aspect, the formulation includes an antigen-binding polypeptide (e.g., an anti Aβ antibody), mannitol, histidine and methionine. In yet another aspect, the formulation includes an Aβ binding polypeptide (e.g., an anti Aβ antibody), mannitol, histidine, methionine and polysorbate 80. In a particular aspect of the invention, the formulation includes about 20 mg/ml an Aβ binding polypeptide (e.g., an anti Aβ antibody), 10 mM histidine, 10 mM methionine, 4% mannitol and has a pH of about 6. In another aspect of the invention, the formulation includes about 20 mg/ml Aβ binding polypeptide (e.g., anti Aβ antibody), 10 mM histidine, 10 mM methionine, 4% w/v mannitol, 0.01% w/v polysorbate 80 and has a pH of about 6. In another aspect of the invention, the formulation includes about 20 mg/ml Aβ binding polypeptide (e.g., anti Aβ antibody), 10 mM histidine, 10 mM methionine, 4% w/v mannitol, 0.005% w/v polysorbate 80 and has a pH of about 6.

Exemplary embodiments of the present invention provide concentrated preparations of an antigen-binding polypeptide (e.g., anti Aβ antibody), often useful as bulk drug product. Furthermore, exemplary embodiments of the present invention are stable to freezing, lyophilization and/or reconstitution. Moreover, exemplary embodiments of the present invention are stable over extended periods of time. For example, the formulations of the present invention are stable for at least about 6, 7, 8, 9, 10, 11, 12, 13, 14, 15, 16, 17, 18, 19, 20, 21, 22, 23, 24, 25, 26, 27, 28, 29, or 30 months. In particular embodiments, the formulations of the present invention are stable for at least about 12 months, for at least about 18 months, for at least about 24 months, or for at least about 30 months.

According to the invention, the formulation may be stored at temperatures from about −80° C. to about 40° C., from about 0° C. to about 25° C., from about 0° C. to about 15° C., or from about 0° C. to about 10° C., preferably from about 2° C. to about 8° C. In various embodiments, the formulation may be stored at about 0° C., 1° C., 2° C., 3° C., 4° C., 5° C., 6° C., 7° C., 8° C., 9° C. or 10° C. In a particular embodiment, the formulation is stored at about 5° C. Generally, the formulation is stable and retains biological activity at these ranges. Ranges intermediate to the above recited temperatures, e.g., from about 2° C. to about 17° C., are also intended to be part of this invention. For example, ranges of values using a combination of any of the above recited values as upper and/or lower limits are intended to be included.

The formulations of the present invention are suitable for delivery by a variety of techniques. In certain embodiments, the formulation is administered parenterally, such as intravenously or intramuscularly. Additionally, one may target delivery of the formulation to the brain (e.g., so that the antibody may cross the blood brain barrier) or the spinal fluid. In a particular embodiment, the formulation is administered intravenously.

Effective doses of the formulations of the present invention vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages need to be titrated to optimize safety and efficacy.

For passive immunization with an antibody, exemplary dosages range from about 0.0001 to 100 mg/kg, and more usually from about 0.01 to about 5 mg/kg, about 0.15 mg/kg to about 3 mg/kg, about 0.5 mg/kg to about 2 mg/kg, preferably about 1 mg/kg to about 2 mg/kg of the host body weight. For example dosages can be 1 mg/kg body weight or 20 mg/kg body weight or within the range of 1-20 mg/kg, preferably about 1 mg/kg, about 2 mg/kg, about 5 mg/kg, about 10 mg/kg, or about 15 mg/kg. In other exemplary embodiments, dosages can be at least 0.5 mg/kg (e.g. 0.5, 0.6, 0.7, 0.75, 0.8, 0.9, 1.0, 1.2, 1.25, 1.3, 1.4, 1.5, 1.6, 1.7, 1.75, 1.8, 1.9, or 2.0 mg/kg), at least 0.75 mg/kg, at least 1.25 mg/kg, at least 1.5 mg/kg, at least 1.75 mg/kg, or at least 2 mg/kg. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Antibody is usually administered on multiple occasions. Intervals between single dosages can be weekly, monthly or yearly. Intervals can also be irregular as indicated by measuring blood levels of antibody to Aβ in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies.

The dosage and frequency of administration can vary depending on whether the treatment is prophylactic or therapeutic. In prophylactic applications, formulations containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 0.5 or 1 to about 200 mg/kg of antibody per dose (e.g. 0.5, 1, 1.5, 2, 5, 10, 20, 25, 50, or 100 mg/kg), with dosages of from 5 to 25 mg/kg being more commonly used) at relatively short intervals is sometimes required until progression of the disease is reduced or terminated, and preferably until the patient shows partial or complete amelioration of symptoms of disease. Thereafter, the patent can be administered a prophylactic regime.

It may be useful to provide the formulations of the invention in dosage unit form for ease of administration and uniformity of dosage. Formulations of the invention may be presented in capsules, ampules or in multi-dose containers. The unit dosage form may comprise any formulation described herein including suspensions, solutions or emulsions of the active ingredient together with formulating agents such as suspending, stabilizing and/or dispersing agents. In an exemplary embodiment, the pharmaceutical dosage unit form may be added to or reconstituted in an intravenous drip bag (e.g. a 50 ml, 100 ml, or 250 ml, or 500 ml drip bag) with a suitable diluent, e.g., sterile pyrogen-free water or saline solution, before administration to the patient, for example, by intravenous infusion. Some pharmaceutical unit dosage forms may require reconstitution with a suitable diluent prior to addition to an intravenous drip bag, particularly lyophilized forms. In exemplary embodiments, the pharmaceutical unit dosage form is a container containing a formulation described herein. The term “container” refers to something, for example, a holder, receptacle, or vessel, into which an object or liquid can be placed or contained, for example, for storage. For example, the container may be a 10 mL glass, type I, tubing vial. Generally, the container should maintain the sterility and stability of the formulation. For example, the vial may be closed with a serum stopper. Furthermore, in various embodiments, the container should be designed so as to allow for withdrawal of 100 mg of formulation or active ingredient (e.g., for single use). Alternatively, the container may be suitable for larger amounts of formulation or active ingredient, for example, from about 10 mg to about 5000 mg, from about 100 mg to about 1000 mg, and from about 100 mg to about 500 mg, about 40 mg to about 250 mg, about 60 mg to about 80 mg, about 80 mg to about 120 mg, about 120 mg to about 160 mg, or ranges or intervals thereof, e.g., about 100 mg to about 200 mg. Ranges intermediate to the above recited amounts, e.g., from about 25 mg to about 195 mg, are also intended to be part of this invention. For example, ranges of values using a combination of any of the above recited values as upper and/or lower limits are intended to be included. In a particular embodiment, the formulation often is supplied as a liquid in unit dosage form.

In another aspect, the present invention provides a kit including a pharmaceutical dosage unit form (for example, a container with a formulation disclosed herein), and instructions for use. Accordingly, the container and the kit may be designed to provide enough formulation for multiple uses. In various embodiments, the kit may further include diluent. The diluent may include excipients, separate or combined. For example, the diluent may include a tonicity modifier such as mannitol, a buffering agent such as histidine, a stabilizer such as polysorbate 80, an anti-oxidant such as methionine, and/or combinations thereof. The diluent may contain other excipients, for example, lyoprotectant, as deemed necessary by one skilled in the art.

Additional useful embodiments of the invention are set forth in the section of this application entitled “Summary of the Invention”.

This invention is further illustrated by the following examples which should not be construed as limiting. The contents of all references, patents and published patent applications cited throughout this application, as well as the figures, are incorporated herein by reference.

### EXAMPLES

Throughout the examples, the following materials and methods were used unless otherwise stated.

Materials and Methods

In general, the practice of the present invention employs, unless otherwise indicated, conventional techniques of chemistry, molecular biology, recombinant DNA technology, immunology (especially, e.g., antibody technology), and standard techniques of polypeptide preparation. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Example 1

### Cloning and Expression of a Therapeutic Polypeptide

In this example, the cloning an expression of a therapeutic polypeptide, in particular, an antigen-binding polypeptide, that is, an antibody capable of binding Aβ, is described.

An exemplary antibody for formulation according to the methods of the instant invention is 3D6. The 3D6 mAb is specific for the N-terminus of Aβ and has been shown to mediate phagocytosis (e.g., induce phagocytosis) of amyloid plaque 3D6 does not recognize secreted APP or full-length APP, but detects only Aβ species with an amino-terminal aspartic acid. Therefore, 3D6 is an end-specific antibody. The cell line designated RB96 3D6.32.2.4 producing the antibody 3D6 has the ATCC accession number PTA-5130, having been deposited on Apr. 8, 2003. The cloning, characterization and humanization of 3D6 antibody is described in U.S. Patent Application Publication No. 20030165496 A1.

Briefly, humanization of the anti Aβ peptide murine monoclonal antibody (designated as m3D6) was carried out by isolating the DNA sequences for m3D6 light chain and heavy chain variable regions (V_{L} and V_{H}) by reverse transcription—polymerase chain reaction (RT-PCR). Based on the determined m3D6 v_{L} and v_{H} DNA sequences, homologous human framework regions were identified. To insure that the humanized antibody retained the ability to interact with the Aβ peptide antigen, critical murine v_{L} and v_{H} framework residues were retained in the humanized 3D6 sequence to preserve the overall structure of the constant domain regions (CDRs) in the context of human kappa light chain and IgG₁ heavy chain sequences. DNA sequences encoding the humanized 3D6 V_{L} and V_{H} sequences identified by this process (including the 5′ signal peptide sequence and 3′ intron splice-donor sequence) were generated by annealing synthesized overlapping DNA oligonucleotides followed by DNA polymerase fill-in reactions. The integrity of each of the humanized variable region sequences was verified by DNA sequencing. FIG. 1 depicts a schematic representation of the predicted structure of an exemplary humanized anti-Aβ peptide 3D6 antibody termed h3D6v2. FIG. 2 identifies the complete amino acid sequences of the h3D6v2 light and heavy chains.

Humanized 3D6 antibody was expressed by transfection of a Chinese Hamster Ovary (CHO) host cell lineage with expression plasmids encoding anti-Aβ antibody light chain and heavy chain genes. CHO cells expressing the antibody were isolated using standard methotrexate—based drug selection/gene amplification procedures. A clonal CHO cell line exhibiting the desired productivity and growth phenotypes was selected and used to establish an antibody expressing cell line using chemically defined medium free of animal or human-derived components.

### Example 2

### Preparation of a Therapeutic Polypeptide Using a Large Scale Bioreactor

In this example, the preparation of therapeutic polypeptide, in particular, an anti-Aβ antibody, is described.

The polypeptide manufacturing process began with the thawing of a starter culture of clonal cells stably expressing the anti-Aβ antibody. Cells were cultured using a chemically defined medium containing no animal or human-derived proteins. Cultures were then expanded and used to inoculate a seed bioreactor, which in turn was used to inoculate multiple production bioreactor cycles. The production bioreactor was operated in fed-batch mode. At the end of the production cycle, the conditioned medium harvest was clarified by microfiltration in preparation for further downstream processing.

The purification processes consisted of standard chromatographic steps followed by filtration. Purified antibody was concentrated by ultrafiltration and diafiltered into formulation buffer absent polysorbate-80. Optionally, polysorbate 80 (vegetable derived) is added to the ultrafiltration/diafiltration retentate pool, followed by bacterial retention filtration. The drug substance was stored frozen at −80° C. and held for further manufacture into drug product, including stabilized liquid formulations described herein.

### Example 3

### Preparation of a Stabilized Liquid Polypeptide Formulation

In this example, a typical composition of a stabilized liquid polypeptide formulation, is described.

Two batches of antibody drug product were manufactured. An initial batch was manufactured by compounding drug substance into an animal and human protein-free formulation containing 20 mg anti Aβ antibody active substance per mL, 10 mM histidine, 10 mM methionine, 4% mannitol, 0.005% polysorbate-80, pH 6.0. The drug product was aseptically filled into vials, at 100 mg anti Aβ antibody active substance/vial. The finished drug product vial contained no preservative and was intended for single-use only.

A second batch of drug product was manufactured by a similar method using a formulation buffer without polysorbate-80.

### Example 4

### Analysis of Stabilized Liquid Polypeptide Formulations

In this example, the analysis of various stabilized liquid polypeptide formulations, is described.

The stability and, in particular, the physicochemical integrity (such as aggregation and deamidation) of the formulation were assessed by the following methods well known in the art: appearance; pH; protein concentration (A280); ELISA, in part, as a test of bioactivity; sodium dodecyl sulfate polyacrylamide gel electrophoresis (SDS-PAGE), in part as a test of aggregation; size exclusion high performance liquid chromatography (SEC-HPLC), in part, as a test of aggregation and stability in general; cation exchange high performance liquid chromatography (CEX-HPLC), in part, as a test of amination and stability in general; and peptide mapping. These methods assessed the recovery and integrity of the protein under test conditions at various temperatures.

Appearance analysis of the formulations was conducted in order to determine the quality of the formulations at various time points. Analysis was conducted based on visual inspection for clarity, color and the presence of particulates. For example, the degree of opalescence was analyzed in terms of reference suspensions. Appearance analysis of the formulations made with and without polysorbate 80 in accordance with the present invention demonstrated that both formulations were acceptable when stored at each of −80° C., 5° C., 25° C., and 40° C. at each of the following timepoints: initial, 1 month, 2 months, 3 months, 6 months, 9 months, and 12 months.

pH analysis sought to determine the maintenance of the formulation's pH within an acceptable range of about 5.5 to about 6.5. pH analysis of formulations made with and without polysorbate 80 in accordance with the present invention demonstrated that both formulations were acceptable when stored at each of −80° C., 5° C., 25° C., and 40° C. at each of the following timepoints: initial, 1 month, 2 months, 3 months, 6 months, 9 months, and 12 months. Generally, the pH never ranged below 5.8 or above 6.2.

Protein concentration analysis by A280 assays was performed to determine the maintenance of the formulation's protein concentration within an acceptable range of about 17 mg/ml to about 23 mg/ml. Protein concentration analysis of formulations made with and without polysorbate 80 in accordance with the present invention demonstrated that both formulations were generally acceptable when stored at each of −80° C., 5° C., 25° C., and 40° C. at each of the following timepoints: initial, 1 month, 2 months, 3 months, 6 months, 9 months, and 12 months. With the exception of the protein concentrations ranging slightly above 23 mg/ml for the formulation without polysorbate 80 when stored at 5° C., 25° C., and 40° C. at the 3 month timepoints, the protein concentration otherwise remained within the acceptable ranges. Accordingly, the protein concentration analysis demonstrated no detectable loss of protein occurring, even at accelerated conditions, particularly for the formulations with polysorbate 80. Moreover, protein concentration generally failed to demonstrate a significant time or temperature dependent change subsequent to the initial time point.

Maintenance of biological activity was assayed, in part, by ELISA techniques. Biological activity was analyzed as BU/mg with acceptable activity being ≧2500 BU/mg or 50% (i.e., 5000 BU/mg equates to 100%). ELISA analysis of formulations made with and without polysorbate 80 in accordance with the present invention demonstrated that both formulations were generally acceptable when stored at each of −80° C., 5° C., 25° C., and 40° C. at each of the following timepoints: initial, 1 month, 2 months, 3 months, 6 months, 9 months, and 12 months. With the exception of the biological activity ranging slightly below 50% at the 12 month time point for both formulations when stored at 40° C., the biological activity otherwise remained within the acceptable ranges.

SEC-HPLC analysis was conducted as a test of aggregation, purity and stability in general. SEC-HPLC runs under conditions using mobile phase chromatography with a sodium phosphate dibasic buffer indicated the formulation was acceptable if the SEC-HPLC analysis identified ≧90% IgG monomer, compared to percentage of high molecular weight product and low molecular weight product. SEC-HPLC analysis of formulations made with and without polysorbate 80 in accordance with the present invention demonstrated that both formulations were generally acceptable when stored at each of −80° C., 5° C., 25° C., and 40° C. at each of the following timepoints: initial, 1 month, 2 months, 3 months, 6 months, 9 months, and 12 months. With the exception of the percentage monomer ranging below 90% for both formulations when stored at 40° C. at each time point at and after 6 months (where the analysis identified greater than at least 10% low molecular weight product for both formulations at each time point), percentage monomer was otherwise within the acceptable range. SEC-HPLC analysis generally demonstrated that although the high molecular weight and low molecular weight profiles were different over time in samples with and without polysorbate, the monomeric form of the antibody generally remained constant, for example at the 12 month time point, when the formulation was stored at 5° C.

CEX-HPLC analysis was conducted as a test of amination and stability in general. CEX-HPLC runs under conditions using mobile phase chromatography with a NaCl buffer produced elution profile and retention times of predominant peaks which were analyzed as being comparable or not comparable to reference standard profiles. CEX-HPLC analysis of formulations made with and without polysorbate 80 in accordance with the present invention demonstrated that both formulations were generally acceptable when stored at each of −80° C., 5° C., 25° C., and 40° C. at each of the following timepoints: initial, 1 month, 2 months, 3 months, 6 months, 9 months, and 12 months. With the exception of the elution profile and retention time of the predominant peaks not being comparable for both formulations when stored at 40° C. at each time point at and after 3 months, the predominant peaks were otherwise comparable to the reference peaks.

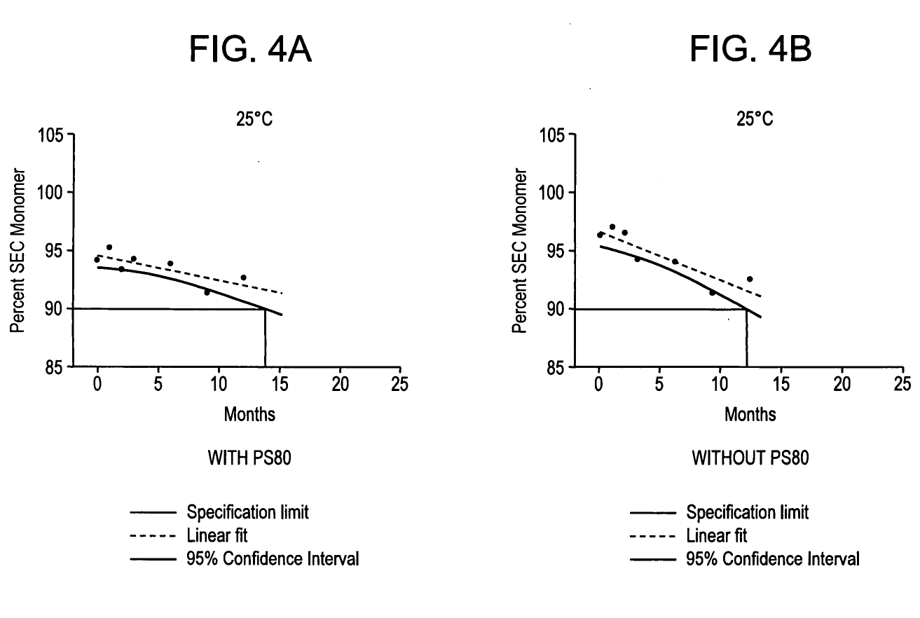

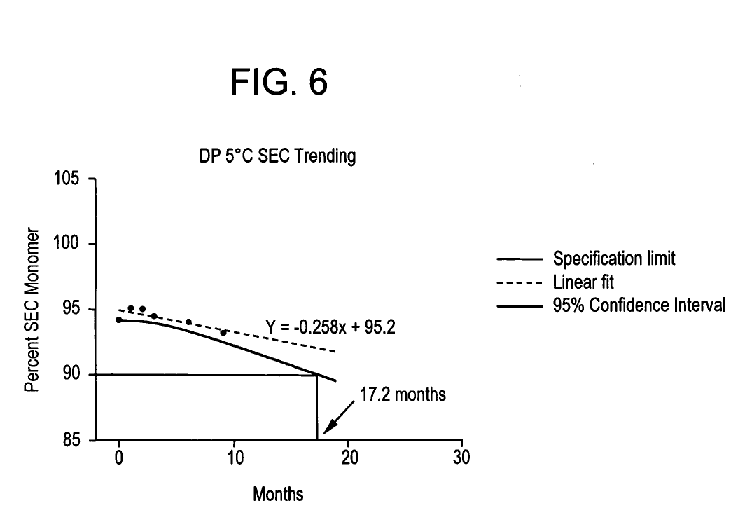

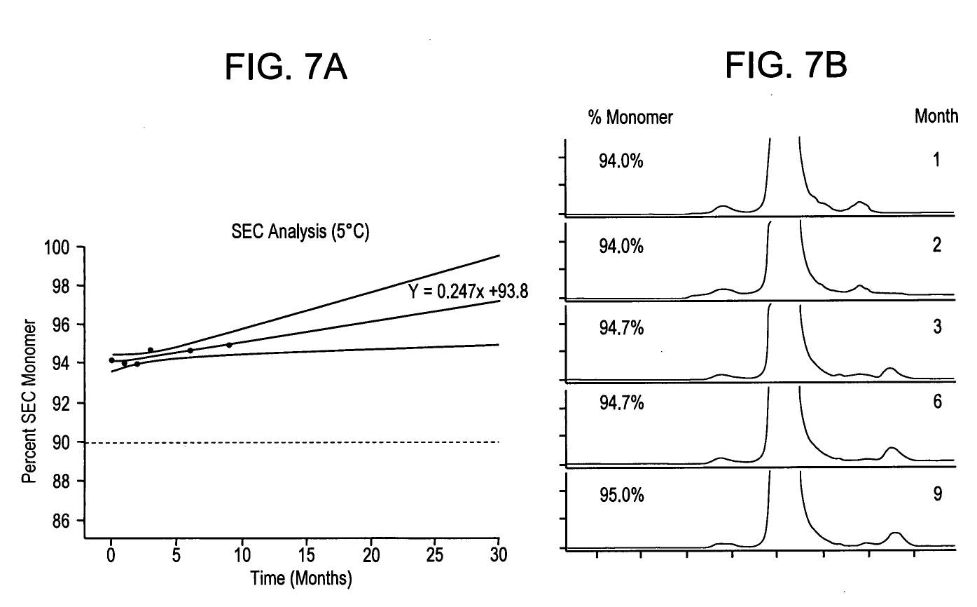

Generally, analysis of the formulations with polysorbate 80 stored at 5° C. allow for the following particularly important conclusions: 1) opalescence, pH, ELISA, CEX-HPLC, SEC-HPLC and SDS PAGE analysis all showed minimal changes in the formulation over 9 months; 2) formulations stored at 5° C. appeared more like reference samples over 9 months than the accelerated samples; 3) peptide mapping showed changes at 5° C.; and 4) SEC-HPLC trending data at 5° C. predicted at least 17.2 months of stability (see FIG. 6), however, upon removing column, instrument and buffer variability, the data allowed for a prediction of greater than 30 months of stability (see FIG. 7). Additionally, accelerated samples with polysorbate 80 stored at 25° C. passed all specifications at 9 months (FIG. 4).

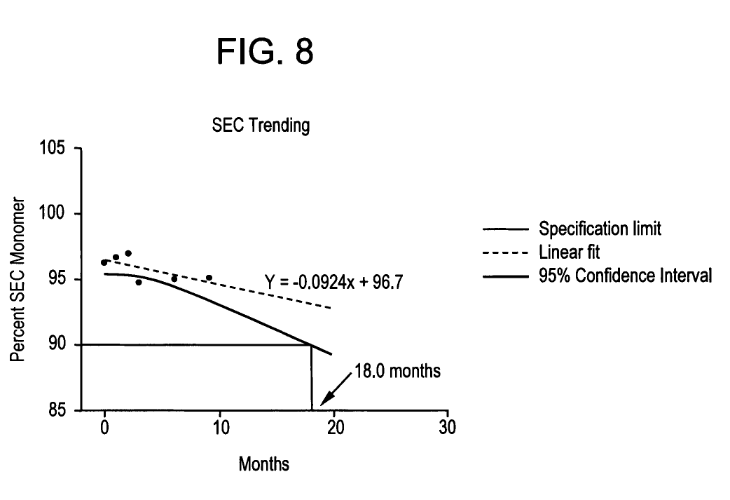

Moreover, analysis of the formulations without polysorbate 80 stored at 5° C. allow for the following particularly important conclusions: 1) opalescence, pH and ELISA analysis all showed minimal changes in the formulation over 9 months; 2) results of the CEX-HPLC and SDS PAGE showed comparable findings to reference samples or the −80° C. control at 9 months; 3) SEC-HPLC analysis showed minor changes over 9 months while changes were more pronounced at accelerated temperatures; and 4) SEC-HPLC trending data predicted at least 18 months of stability, even with assay variability issues (see FIG. 8).

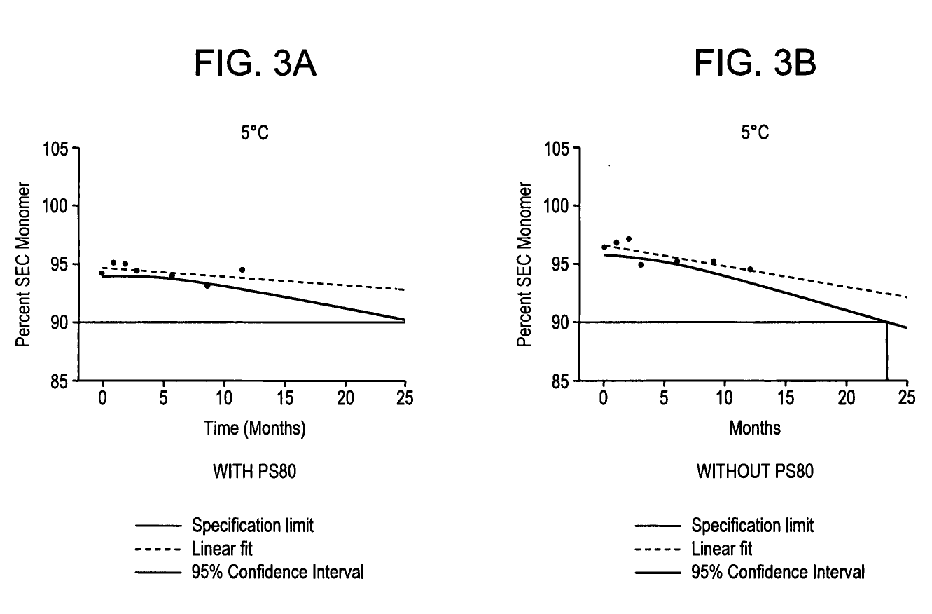

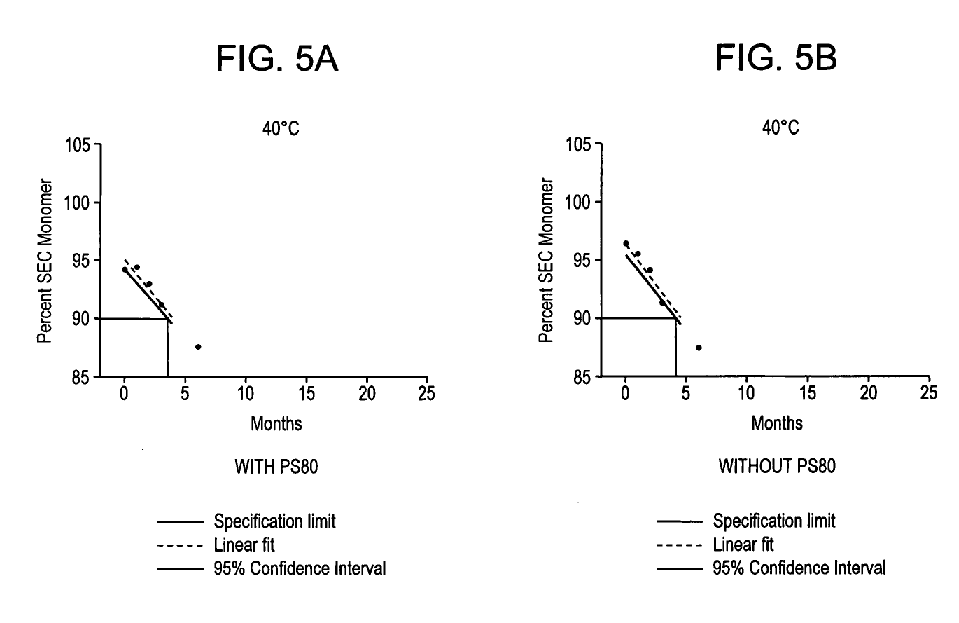

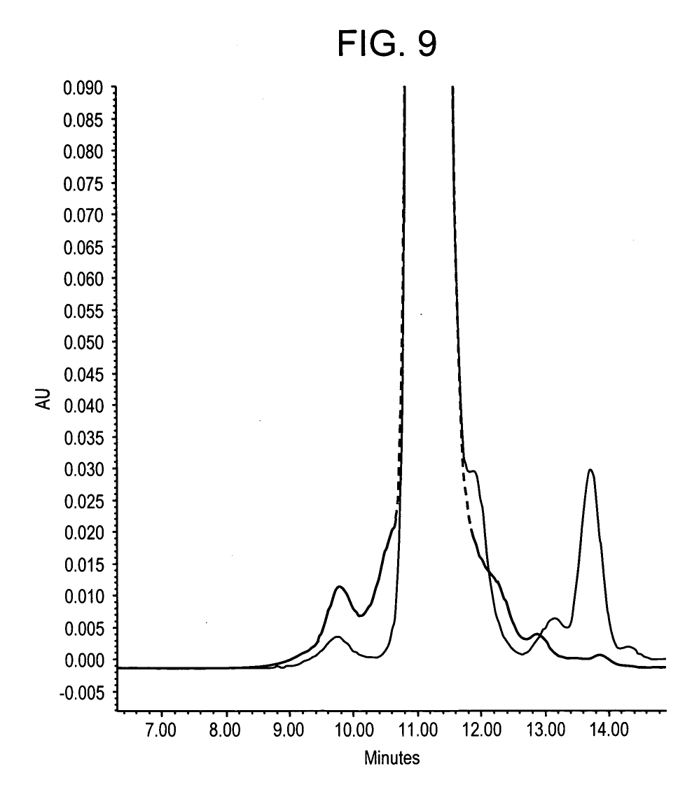

FIGS. 3-5 are graphical depictions of the shelf life predictions for the formulations (with and without PS80) made in accordance with the present invention and stored at 5° C., 25° C., and 40° C., respectively. Generally, FIGS. 3-5 indicate that storage of the formulations of the present invention at higher temperatures reduces the expected shelf life. FIG. 3, in particular, indicates that the formulation has an expected shelf life of at least 18 months when the formulation is stored at 5° C. FIG. 4 indicates that storage of the formulation at room temperature (25° C.) may serve to reduce expected shelf life to about 12 months. FIG. 5 further demonstrates that storage of the formulation at 40° C. may serve to reduce expected shelf life to about 4 months. Still further, FIG. 9 indicates that at, for example, 5° C. at 12 months, PS80 reduces the presence of high molecular weight by-products, for example, polypeptide aggregates.

### Example 5

### Stability Studies on Use of Methionine as an Anti-Oxidant

In this example, the analysis of various liquid polypeptide formulations stabilized with an antioxidant, in particular, methionine, is described.

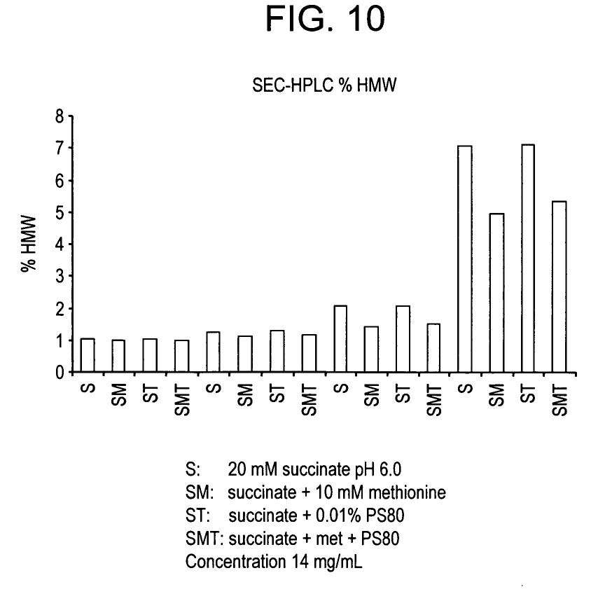

Studies were conducted to determine the effect of methionine on maintaining the stability of an antibody in a therapeutic antibody formulation. SEC-HPLC analysis was conducted over 6 months at various temperatures on four antibody (an anti-CD22 IgG₄ antibody) samples: an antibody formulation with 20 mM succinate at a pH of 6.0; an antibody formulation with 20 mM succinate and 10 mM methionine; an antibody formulation with 20 mM succinate and 0.01% PS80; and an antibody formulation with 20 mM succinate, 10 mM methionine and 0.01% PS80. Generally, the results indicated that methionine desirably lessens high molecular weight (HMW) formation, for example, the formation of aggregates. Moreover, methionine decreases temperature dependent increase in the percent of HMW (see FIG. 10).

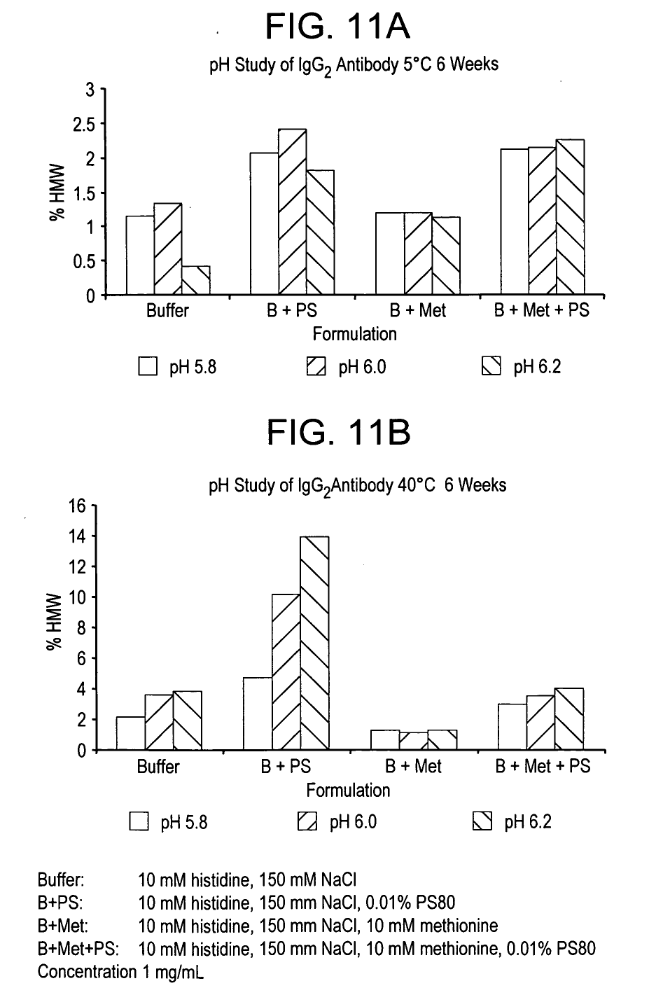

Furthermore, a pH stability study (at pH 5.8, 6.0 and 6.2) was conducted over 6 weeks at various temperatures (5° C. and 40° C.) on the following four antibody (an anti-B7.2 IgG₂ antibody) samples: (1) a sample including antibody, 10 mM histidine and 150 mM NaCl; (2) a sample including antibody, 10 mM histidine, 150 mM NaCl and 0.01% PS80; (3) a sample including antibody, 10 mM histidine, 150 mM NaCl and 10 mM methionine; and (4) a sample including antibody, 10 mM histidine, 150 mM NaCl, 10 mM methionine and 0.01% PS80. SEC-HPLC analysis was conducted. The results demonstrated that methionine decreases the temperature dependent increase in percent of by-product formation (e.g., HMW by-products) over the indicated pH range, (see FIG. 11). As shown in FIG. 11, samples containing methionine displayed a low amount of aggregation when maintained at 40° C. for six weeks, which was similar to that for samples maintained at 5° C. for six weeks.

### Example 6

### Excipient Analysis of Stabilized Liquid Polypeptide Formulations Using Differential Scanning Calorimetry

In this example, excipient analysis of various liquid polypeptide formulations using differential scanning calorimetry, is described.

A primary goal of protein drug formulation is to stabilize a protein in its native, biologically active form. Typically this can be done by screening various excipients in a base formulation and monitoring their effect on the molecule's molecular weight and activity. These parameters are indicative of stability. Another measurement of stability is thermal denaturation which can be monitored using a variety of biophysical techniques. Generally, increased levels of protein stability have been attributed to high melting, denaturation or decomposition temperatures. Accordingly, thermal properties of an exemplary antigen-binding polypeptide, in particular, an IgG1 monoclonal antibody were monitored in the presence of various excipients using a VP-Capillary Differential Scanning Calorimeter. Specifically, the apparent Tₘs were determined for formulations containing 10 mM histidine (pH 6.0) with various excipients. Several excipients were shown to provide increased or decreased thermal stability. Because increased levels of protein stability have been attributed to a high melting temperature, excipients in samples imparting an increased Tₘ2 or Tₘ3, as compared to control Tₘ2/Tₘ3 values (respectively, 74.9° C. and 83.4° C.), were deemed to be especially desirable excipients (see Table 1 below).

Accordingly, it was concluded that excipients such as glucose (formulated at a concentration of 4% and 10%), sucrose (formulated at a concentration of 4% and 10%), sorbitol (formulated at a concentration of 4% and 10%), and mannitol (formulated at a concentration of 4% and 10%), performed especially well in stabilizing a liquid polypeptide formulation, in particular, an antibody IgG formulation.

TABLE 1
Excipient Analysis Results

| Excipient | Concentration | | Tₘ1* | Tₘ2* | Tₘ3* |
|---|---|---|---|---|---|
| Histidine | 10 | mM | — | 74.9 | 83.4 |
| (Control) | | | | | |
| NaCl | 10 | mM | 69.3 | 74.8 | 82.9 |
| | 100 | mM | 67.9 | 74.4 | 82.4 |
| | 500 | mM | 66.5 | 74.5 | 81.9 |
| | 1 | M | 65.4 | 74.9 | 82.3 |
| CaCl2 | 10 | mM | 68.7 | 74.6 | 82.7 |
| | 100 | mM | 68.5 | 74.5 | 82.4 |
| Methionine | 30 | mM | — | 74.5 | 83.7 |
| Vitamin C | ˜30 | mM | 52.2 | 68.7 | — |

| | | | | |
|---|---|---|---|---|
| Polysorbate 20 | 0.005% | — | 74.5 | 83.7 |
| | 0.01% | — | 74.5 | 83.8 |
| | 0.1% | — | 74.4 | 83.7 |
| Polysorbate 80 | 0.005% | — | 74.6 | 83.8 |
| | 0.01% | — | 74.5 | 83.7 |
| | 0.1% | — | 74.5 | 83.7 |
| Glucose | 0.5% | — | 74.7 | 83.8 |
| | 2% | — | 74.9 | 83.9 |
| | 4% | — | 75.0 | 84.3 |
| | 10% | — | 75.8 | 84.9 |
| Sucrose | 0.5% | — | 74.6 | 83.6 |
| | 2% | — | 74.8 | 83.8 |
| | 4% | — | 75.0 | 83.9 |
| | 10% | — | 75.5 | 84.4 |
| Sorbitol | 0.5% | — | 74.8 | 83.6 |
| | 2% | — | 75.0 | 83.8 |
| | 4% | — | 75.2 | 84.1 |
| | 10% | — | 75.9 | 84.8 |
| Mannitol | 0.5% | — | 74.8 | 83.6 |
| | 2% | — | 74.9 | 83.8 |
| | 4% | — | 75.2 | 84.1 |
| | 10% | — | 75.9 | 84.8 |

*In the control (10 mM histidine, pH 6.0) two transitions were observed, Tₘ2 and Tₘ3. An earlier transition (Tₘ1) was seen in the presence of some excipients.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A liquid formulation comprising,
a therapeutically active antigen-binding polypeptide, wherein the polypeptide exhibits by-product formation during storage, and
an antioxidant, wherein said antioxidant is present in an amount sufficient to reduce the by-product formation of the polypeptide during storage of the formulation.

2. The formulation of claim 1, wherein the therapeutically active antigen-binding polypeptide component is selected from the group consisting of an antibody, an antibody Fv fragment, an antibody Fab fragment, an antibody Fab′(2) fragment, an antibody Fd fragment, a single-chain antibody (scFv), a single domain antibody fragment (Dab), a beta-pleated sheet polypeptide comprising at least one antibody complementarity determining region (CDR), and a non-globular polypeptide comprising at least one antibody complementarity determining region.

3. The formulation of claim 1, wherein the therapeutically active antigen-binding polypeptide is an antibody.

4. The formulation of claim 3, wherein the antibody is of a subtype selected from the group consisting of IgG1, IgG2, IgG3, and IgG4.

5. The formulation of claim 3, wherein the by-product is selected from the group consisting of a high molecular weight polypeptide aggregate, a low molecular weight polypeptide degradation product, and combinations thereof.

6. The formulation of claim 5, wherein the high molecular weight aggregate is selected from the group consisting of antibody:antibody complexes, antibody:antibody fragment complexes, antibody fragment:antibody fragment complexes, and combinations thereof.

7. The formulation of claim 5, wherein the low molecular weight polypeptide degradation product is selected from the group consisting of an antibody light chain, an antibody heavy chain, an antibody light chain and heavy chain complex, an antibody fragment, and combinations thereof.

8. The formulation of claim 1, wherein the antioxidant is selected from the group consisting of methionine and an analog thereof.

9. The formulation of claim 8, wherein the methionine is present in an amount of about 0.1 mM to about 25 mM.

10. The formulation of claim 8, wherein the methionine is present in an amount of about 10 mM.

11. The formulation of claim 1, wherein the formulation is suitable for administering parenterally, intravenously, intramuscularly, subcutaneously, intracranially, or epidurally.

12. The formulation of claim 1, wherein the formulation is capable of traversing the blood-brain-barrier.

13. The formulation of claim 1, wherein the formulation further comprises a tonicity agent.

14. The formulation of claim 13, wherein the tonicity agent is mannitol.

15. The formulation of claim 13, wherein the formulation is suitable for intravenous administration.

16. The formulation of claim 1, wherein the formulation further comprises histidine.

17. A liquid formulation comprising an antigen-binding polypeptide, methionine, histidine, and mannitol.

18. The formulation of claim 17, wherein the formulation is suitable for intravenous administration.

19. The formulation of any one of the preceding claims, wherein the antigen-binding polypeptide binds to an antigen of an antigen class selected from the group consisting of cancer antigens, autoimmune antigens, allergens, and pathogens.

20. The formulation of any one of the preceding claims, wherein the antigen-binding polypeptide is present from about 0.1 mg/ml to about 200 mg/ml.

21. The formulation of any one of the preceding claims, wherein antigen-binding polypeptide is present at about 17 mg/ml.

22. The formulation of any one of claims 1-20, wherein antigen-binding polypeptide is present at about 20 mg/ml.

23. The formulation of any one of claims 1-20, wherein antigen-binding polypeptide is present at about 30 mg/ml.

24. The formulation of any one of claims 14 and 17-23, wherein mannitol is present in amount sufficient to maintain isotonicity of the formulation.

25. The formulation of any one of claims 14 and 17-24, wherein mannitol is present from about 2% w/v to about 10% w/v.

26. The formulation of any one of claims 14 and 17-24, wherein mannitol is present at about 4% w/v.

27. The formulation of any one of claims 14 and 17-24, wherein mannitol is present at about 6% w/v.

28. The formulation of any one of claims 14 and 17-24, wherein mannitol is present at about 10% w/v.

29. The formulation of any one of claims 16-28, wherein histidine is present in an amount sufficient to maintain a physiologically suitable pH.

30. The formulation of any one of claims 16-29, wherein histidine is present from about 0.1 mM to about 25 mM.

31. The formulation of any one of claims 16-29, wherein histidine is present at about 10 mM.

32. The formulation of any one of the preceding claims, further comprising a stabilizer.

33. The formulation of claim 32, wherein the stabilizer comprises polysorbate 80.

34. The formulation of claim 33, wherein the polysorbate 80 is present from about 0.001% w/v to about 0.01% w/v.

35. The formulation of claim 33, wherein the polysorbate 80 is present at about 0.005% w/v.

36. The formulation of claim 33, wherein the polysorbate 80 is present at about 0.01% w/v.

37. The formulation of any one of the preceding claims, wherein the formulation has a pH of about 4 to about 9.

38. The formulation of any one of the preceding claims, wherein the formulation has a pH of about 6 to about 7.

39. The formulation of any one of the preceding claims, wherein the formulation is stable to freezing.

40. The formulation of any one of the preceding claims, wherein the formulation is stable for at least about 12 months.

41. The formulation of any one of the preceding claims, wherein the formulation is stable for at least about 18 months.

42. The formulation of any one of the preceding claims, wherein the formulation is stable for at least about 24 months.

43. The formulation of any one of the preceding claims, wherein the formulation is stable for at least about 30 months.

44. The formulation of any one of the preceding claims, wherein the formulation is stable at about −80° C. to about 40° C.

45. The formulation of any one of the preceding claims, wherein the formulation is stable at about 0° C. to about 25° C.

46. The formulation of any one of the preceding claims, wherein the formulation is stable at about 2° C. to about 8° C.

47. A pharmaceutical unit dosage form comprising an effective amount of the formulation of any of the preceding claims for treating disease in a patient via administration of said dosage form to said patient.

48. The pharmaceutical unit dosage form of claim 47 which is a container containing said formulation.

49. The container of claim 47, which is a vial containing about 1 mg to about 2000 mg of said Aβ binding polypeptide.

50. The container of claim 47, which is a vial containing about 50 mg to about 1500 mg of said Aβ binding polypeptide.

51. The container of claim 47, which is a vial containing about 5 mg to about 50 mg of said Aβ binding polypeptide.

52. The pharmaceutical unit dosage form of claim 47, wherein said vial has a volume of about 2 to about 100 ml.

53. The pharmaceutical unit dosage form of claim 47, wherein said vial has a volume of about 2 to about 10 ml.

54. The pharmaceutical unit dosage form any of claims 47-53, suitable for intravenous infusion to said patient.

55. A kit comprising,
a) the pharmaceutical unit dosage form of any one of claims 47-54; and
b) instructions for use.

56. A container comprising the pharmaceutical unit dosage form of claim 47 which is a container labeled for use.

57. The container of claim 56 labeled for prophylactic use.

58. The container of claim 56 labeled for therapeutic use.

59. A method for increasing the stability of an antigen-binding polypeptide in a liquid pharmaceutical formulation, where the polypeptide exhibits by-product formation during storage in a liquid formulation, the method comprising incorporating into the formulation an anti-oxidant in an amount sufficient to reduce the amount of by-product formation of the polypeptide.

60. The method of claim 59, wherein the antigen-binding polypeptide component is selected from the group consisting of an antibody, an antibody Fv fragment, an antibody Fab fragment, an antibody Fab′(2) fragment, an antibody Fd fragment, a single-chain antibody (scFv), a single domain antibody fragment (Dab), a beta-pleated sheet polypeptide comprising at least one antibody complementarity determining region (CDR), and a non-globular polypeptide comprising at least one antibody complementarity determining region.

61. The method of claim 59, wherein the by-product is selected from the group consisting of a high molecular weight polypeptide aggregate, a low molecular weight polypeptide degradation product, and combinations thereof.

62. The method of claim 59, wherein the antioxidant is selected from the group consisting of methionine and an analog thereof.

63. A method for preparing the formulation of any of claims 1-46, comprising combining the excipients of the formulation.

64. A method for preparing the formulation of any of claims 1-46, comprising combining the antigen binding polypeptide with one or more diluents, wherein said one or more diluents comprise the excipients of the formulation.

65. A method for preparing a pharmaceutical unit dosage form comprising combining the formulation of any of claims 1-46 in a suitable container.

66. A method for preparing the formulation of any one of claims 1-46 comprising combining a solution comprising the antigen binding polypeptide and a least a portion of the excipients with a diluent comprising the remainder of the excipients.

67. A formulation stable for at least about 12 months at a temperature of above freezing to about 10° C. and having a pH of about 5.5 to about 6.5, comprising:
i. at least antigen-binding polypeptide at a concentration of about 1 mg/ml to about 30 mg/ml;
ii. mannitol at a concentration of about 4% w/v or NaCl at a concentration of about 150 mM;
iii. about 5 mM to about 10 mM histidine or succinate; and
iv. 10 mM methionine.

68. The formulation of claim 67, wherein the formulation is stable for at least about 24 months at a temperature of about 2° C. to 8° C., and comprises polysorbate 80 at a concentration of about 0.001% w/v to about 0.01% w/v.

69. The formulation of claim 67, wherein the formulation has a pH of about 6.0 to about 6.5 and comprises about 10 mg/ml antigen-binding polypeptide, about 10 mM histidine and about 4% w/v mannitol and about 0.005% w/v polysorbate 80.

70. The formulation of claim 67, wherein the formulation has a pH of about 6.0 to about 6.2 and comprises about 20 mg/ml antigen-binding polypeptide, about 10 mM histidine, about 4% w/v mannitol and about 0.005% w/v polysorbate 80.

71. The formulation of claim 67, wherein the formulation has a pH of about 6.0 to about 6.2 and comprises about 30 mg/ml antigen-binding polypeptide, about 10 mM histidine, about 4% w/v mannitol and about 0.005% w/v polysorbate 80.

72. The formulation of claim 71, further comprising about 4% w/v mannitol.

73. The formulation of claim 71, further comprising polysorbate 80 at a concentration of about 0.001% w/v to about 0.01% w/v.

74. The formulation of claim 73, comprising about 0.005% w/v polysorbate 80.

75. The formulation of claim 71, wherein the antigen-binding polypeptide is present at a concentration of about 17 mg/ml to about 23 mg/ml.

76. A formulation stable for at least about 24 months at a temperature of about 2° C. to about 8° C. and having a pH of about 5.5 to about 6.5, comprising about 2 mg/ml to about 23 mg/ml of a antigen-binding polypeptide, about 10 mM succinate, about 10 mM methionine, about 4% w/v mannitol and about 0.005% w/v polysorbate 80.

77. A formulation stable when thawed from about −50° C. to about −80° C., comprising about 40 to about 60 mg/ml of antigen-binding polypeptide, about 1.0 mg/ml to about 2.0 mg/ml histidine, about 1.0 mg/ml to 2.0 mg/ml methionine and about 0.05 mg/ml polysorbate 80, wherein the formulation has a pH of about 6.0.

78. The formulation of claim 77, wherein mannitol is excluded.

79. A formulation comprising about 20 mg/mL antigen-binding polypeptide, about 10 mM L-histidine, about 10 mM methionine, about 4% mannitol and having a pH of about 6.

80. A formulation comprising about 30 mg/mL antigen-binding polypeptide, about 10 mM succinate, about 10 mM methionine, about 6% mannitol and having a pH of about 6.2.

81. A formulation comprising about 20 mg/mL antigen-binding polypeptide, about 10 mM L-histidine, about 10 mM methionine, about 4% mannitol, about 0.005% polysorbate 80, and having a pH of about 6.

82. A formulation comprising about 10 mg/mL antigen-binding polypeptide, about 10 mM succinate, about 10 mM methionine, about 10% mannitol, about 0.005% polysorbate 80, and having a pH of about 6.5.

83. A formulation comprising about 5 mg/mL to about 20 mg/mL antigen-binding polypeptide, about 5 mM to about 10 mM L-histidine, about 10 mM methionine, about 4% mannitol, about 0.005% polysorbate 80, and having a pH of about 6.0 to about 6.5.

84. A formulation comprising about 5 mg/mL to about 20 mg/mL antigen-binding polypeptide, about 5 mM to about 10 mM L-histidine, about 10 mM methionine, about 150 mM NaCl, about 0.005% polysorbate 80, and having a pH of about 6.0 to about 6.5.

85. A pharmaceutical unit dosage form, comprising a formulation comprising:
a. about 10 mg to about 250 mg of an antigen-binding polypeptide;
b. about 4% mannitol or about 150 mM NaCl;
c. about 5 mM to about 10 mM histidine or succinate; and
d. about 10 mM methionine

86. The pharmaceutical unit dosage form of claim 85, comprising about 0.001% to about 0.1% polysorbate 80.

87. The pharmaceutical unit dosage form of claim 86, comprising about 40 mg to about 60 mg of the antigen-binding polypeptide.

88. The pharmaceutical unit dosage form of claim 86, comprising about 60 mg to about 80 mg of the antigen-binding polypeptide.

89. The pharmaceutical unit dosage form of claim 86, comprising about 80 mg to about 120 mg of the antigen-binding polypeptide.

90. The pharmaceutical unit dosage form of claim 86, comprising about 120 mg to about 160 mg of the antigen-binding polypeptide.

91. The pharmaceutical unit dosage form of claim 86, comprising about 160 mg to about 240 mg of the antigen-binding polypeptide.

92. A therapeutic product, comprising:
a. a glass vial, comprising a formulation comprising:
i. about 10 mg to about 250 mg of a antigen-binding polypeptide,
ii. about 4% mannitol or about 150 mM NaCl,
iii. about 5 mM to about 10 mM histidine, and
iv. about 10 mM methionine; and
b. labeling for use comprising instructions to use the appropriate volume necessary to achieve a dose of about 0.15 mg/kg to about 5 mg/kg.

93. The therapeutic product of claim 92, wherein the dose is about 0.5 mg/kg to about 3 mg/kg.

94. The therapeutic product of claim 92, wherein the dose is about 1 mg/kg to about 2 mg/kg.

95. The therapeutic product of claim 92, wherein the antigen-binding polypeptide concentration is about 10 mg/ml to about 60 mg/ml.

96. The therapeutic product of claim 92, wherein the antigen-binding polypeptide concentration is about 20 mg/ml.

97. The therapeutic product of claim 92, further comprising about 0.005% polysorbate 80.

98. The therapeutic product of claim 92, wherein the use is a subcutaneous administration.

99. The therapeutic product of claim 92, wherein the use is an intravenous administration.


SEQUENCE LISTING (2 sequences)

SEQ ID NO: 1 - protein, 219 aa, Artificial Sequence
  Synthetic Construct
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, Artificial Sequence
  Synthetic Construct
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG